SEQUENCE LISTING (38 sequences)

SEQ ID NO: 1 - protein, 128 aa, Artificial Sequence
  Description of Artificial Sequence Synthetic polypeptide
AGLMQSGAVMKNSGASVRVSCQADGYDFIDYVIHWFRQRRGEGLEWLGWMNPSGGGTNYPRPFQGKVTMTRDTSTETAYLDVRGLTYDDTAVYYCVRDRANGSGRRRFESVNWFLDLWGRGTQITVVS

SEQ ID NO: 2 - protein, 99 aa, Artificial Sequence
  Description of Artificial Sequence Synthetic polypeptide
QSALTQPRSVSASPGQSVTISCTGTHNYVSWCQQKPGQAPKLLIYDFNKRPSGVSDRFSGSTSGNTASLTISGLQADDEGHYFCWAFENIGGGTKLTVL

SEQ ID NO: 3 - protein, 128 aa, Artificial Sequence
  Description of Artificial Sequence Synthetic polypeptide
SAELVQSGAVVKKPGTSVKVSCQAYGYTFTDYLIHWLRQAPGQGLEWMGWMNPVYGQVNYAQNFQGRVSMTRDIYRETAFLEVRDLKTDDTGTYYCVRDTGDGSRRHFDSINWFLDLWGRGTWIRVAP

SEQ ID NO: 4 - protein, 99 aa, Artificial Sequence
  Description of Artificial Sequence Synthetic polypeptide
QCVLTQPRSVSGSPGQSVTISCTGTHNYVSWCQHHPGNAPKLLLYDFDKRPSGISDRFSGSRSGNTASLTISGLQPEDEADYFCWAFEAFGGGTKVLVL

SEQ ID NO: 5 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCARGPSGENYPFHYWGQGVRVVVSS

SEQ ID NO: 6 - protein, 100 aa, Artificial Sequence
  Description of Artificial Sequence Synthetic polypeptide
ASALTQPASMSASPGQSVTISCSGTRHIISAWFQQYPGKPPKLIIFDDDKRPSGVPSRFSASRPGDTASLTISNVQPEDEATYICNTYEFFGGGTRLTVL

SEQ ID NO: 7 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVQYSWRFEGRVTMTRDMDTETAFMELRGLRVDDTAVYYCARGPSGENYPFHYWGQGVRVVVSS

SEQ ID NO: 8 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVDYSWRFEGRVTMTRDMDTETAFMELRGLRVDDTAVYYCARGPSGENYPFHYWGQGVRVVVSS

SEQ ID NO: 9 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVSYSWRFEGRVTMTRDMDTETAFMELRGLRVDDTAVYYCARGPSGENYPFHYWGQGVRVVVSS

SEQ ID NO: 10 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVAYSWRFEGRVTMTRDMDTETAFMELRGLRVDDTAVYYCARGPSGENYPFHYWGQGVRVVVSS

SEQ ID NO: 11 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVNYAWRFEGRVTMTRDMDTETAFMELRGLRVDDTAVYYCARGPSGENYPFHYWGQGVRVVVSS

SEQ ID NO: 12 - protein, 120 aa, Artificial Sequence
  Description of Artificial Sequence Synthetic polypeptide
HVQLVQSGGGVKKIGAAVRISCEVTGYKFMDQLINWVRQAPGQGLEWMGWMNPTYGQVNYNWRFEGRVTMTRDMDTETAFMELRGLRVDDTAVYYCARGPSGENYPFHYWGQGVRVVVSS

SEQ ID NO: 13 - protein, 10 aa, Artificial Sequence
  Description of Artificial Sequence Synthetic peptide
GYDFIDYVIH

SEQ ID NO: 14 - protein, 17 aa, Artificial Sequence
  Description of Artificial Sequence Synthetic peptide
WMNPSGGGTNYPRPFQG

SEQ ID NO: 15 - protein, 22 aa, Artificial Sequence
  Description of Artificial Sequence Synthetic peptide
VRDRANGSGRRRFESVNWFLDL

SEQ ID NO: 16 - protein, 8 aa, Artificial Sequence
  Description of Artificial Sequence Synthetic peptide
TGTHNYVS

SEQ ID NO: 17 - protein, 7 aa, Artificial Sequence
  Description of Artificial Sequence Synthetic peptide
DFNKRPS

SEQ ID NO: 18 - protein, 5 aa, Artificial Sequence
  Description of Artificial Sequence Synthetic peptide
WAFEN

SEQ ID NO: 19 - protein, 10 aa, Artificial Sequence
  Description of Artificial Sequence Synthetic peptide
GYTFTDYLIH

SEQ ID NO: 20 - protein, 17 aa, Artificial Sequence
  Description of Artificial Sequence Synthetic peptide
WMNPVYGQVNYAQNFQG

SEQ ID NO: 21 - protein, 21 aa, Artificial Sequence
  Description of Artificial Sequence Synthetic peptide
VRDTGDGSRRHFDSINWFLDL

SEQ ID NO: 22 - protein, 7 aa, Artificial Sequence
  Description of Artificial Sequence Synthetic peptide
DFDKRPS

SEQ ID NO: 23 - protein, 5 aa, Artificial Sequence
  Description of Artificial Sequence Synthetic peptide
WAFEA

SEQ ID NO: 24 - protein, 10 aa, Artificial Sequence
  Description of Artificial Sequence Synthetic peptide
GYKFMDQLIN

SEQ ID NO: 25 - protein, 17 aa, Artificial Sequence
  Description of Artificial Sequence Synthetic peptide
WMNPTYGQVNYSWRFEG

SEQ ID NO: 26 - protein, 13 aa, Artificial Sequence
  Description of Artificial Sequence Synthetic peptide
ARGPSGENYPFHY

SEQ ID NO: 27 - protein, 9 aa, Artificial Sequence
  Description of Artificial Sequence Synthetic peptide
SGTRHIISA

SEQ ID NO: 28 - protein, 7 aa, Artificial Sequence
  Description of Artificial Sequence Synthetic peptide
DDDKRPS

SEQ ID NO: 29 - protein, 5 aa, Artificial Sequence
  Description of Artificial Sequence Synthetic peptide
NTYEF

SEQ ID NO: 30 - protein, 127 aa, Artificial Sequence
  Description of Artificial Sequence Synthetic polypeptide
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQS

SEQ ID NO: 31 - protein, 98 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 32 - protein, 10 aa, Artificial Sequence
  Description of Artificial Sequence Synthetic peptide
YYYGSGSYYN

SEQ ID NO: 33 - protein, 17 aa, Artificial Sequence
  Description of Artificial Sequence Synthetic peptide
YWYFDLWGRGTLVTVSS

SEQ ID NO: 34 - protein, 15 aa, Artificial Sequence
  Description of Artificial Sequence Synthetic peptide
YFDYWGQGTLVTVSS

SEQ ID NO: 35 - protein, 99 aa, Artificial Sequence
  Description of Artificial Sequence Synthetic polypeptide
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVL

SEQ ID NO: 36 - protein, 100 aa, Artificial Sequence
  Description of Artificial Sequence Synthetic polypeptide
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTFH

SEQ ID NO: 37 - protein, 94 aa, Artificial Sequence
  Description of Artificial Sequence Synthetic polypeptide
QSALTQPASVSGSPGQSITISCTGTSSDVGSYNLVSWYQQHPGKAPKLMIYEGSKRPSGVSNRFSGSKSGNTASLTISGLQAEDEADYYCCSYA

SEQ ID NO: 38 - protein, 12 aa, Artificial Sequence
  Description of Artificial Sequence Synthetic peptide
WVFGGGTKLTVL

(19) United States
(12) Patent Application Publication
(10) Pub. No.: US 2021/0040185 A1
(43) Pub. Date: Feb. 11, 2021

(54) ANTI-HIV ANTIBODIES

(71) Applicants: ATRECA, INC., South San Francisco, CA (US); UNIVERSITY OF MARYLAND, BALTIMORE, Baltimore, MD (US); The United States of America as Represented by the Department of Veterans Affairs, Washington, DC (US)

(72) Inventors: Guy L. CAVET, Burlingame, CA (US); Anthony Devico, Alexandria, VA (US); Dongkyoon Kim, Redwood City, CA (US); George K. Lewis, Baltimore, MD (US); Shaun M. Lippow, S. San Francisco, CA (US); Mohammad Sajadi, Cockeysville, MD (US)

(21) Appl. No.: 17/015,290

(22) Filed: Sep. 9, 2020

Related U.S. Application Data
(63) Continuation of application No. PCT/US2019/021495, filed on Mar. 8, 2019.
(60) Provisional application No. 62/641,212, filed on Mar. 9, 2018.

(51) Int. Cl. C07K 16/10

(52) U.S. Cl. CPC C07K 16/1045; C07K 2317/565

(57) ABSTRACT

Provided herein are anti-HIV antibodies, compositions comprising such antibodies, and methods of producing the antibodies. Additionally provided are methods of treating or preventing HIV infection, or a complication of HIV infection, using the anti-HIV antibodies.

### CROSS-REFERENCE TO RELATED APPLICATIONS

This application is a continuation of International Patent Application No. PCT/US2019/021495, which claims priority benefit to U.S. provisional patent application No. 62/641,212, filed Mar. 9, 2018, each of which is herein incorporated by reference for all purposes.

### SEQUENCE LISTING

The instant application contains a Sequence Listing which has been submitted electronically in ASCII format and is hereby incorporated by reference in its entirety. Said ASCII copy, created on Sep. 8, 2020, is named SEQTXT 097519-1210173.TXT and is 24,413 bytes in size.

### BACKGROUND OF THE INVENTION

Analysis of HIV-infected individuals has led to discovery of hundreds of antibodies active against many different HIV strains. Various active anti-HIV antibodies identified to date include those that target five major sites of vulnerability on the virus, including the CD4 binding site, the V1-V2 apex, V3 glycans, the membrane proximal external region (MPER) and the gp120-gp41 interface. Highly active antibodies from two lineages (L1 and L2) found in the same donor have recently been characterized. These antibodies include L1A1, L1A4, and L1A2 from lineage L1, and L2A1 from lineage L2.

### BRIEF SUMMARY OF SOME ASPECTS OF THE INVENTION

The present disclosure provides variants of antibodies L1A1, L1A4, and L2A1. In some embodiments, the variants have broadly neutralizing activity. In some embodiments, the variants exhibit reduced immunogenicity and/or enhanced production properties compared to the parent L1 or L2 antibody. Thus, in one aspect, provided herein is an anti-HIV antibody comprising a heavy chain variable (VH) region and a light chain variable (VL) region, wherein: (a) the VH region comprises a CDR1 sequence, a CDR2, sequence, and a CDR3 sequence as set forth in Table 2 for the antibody designated L1A1 in which at least one of CDR2 or CDR3 comprises a substitution, wherein the substitution is selected from the group consisting of Y or F at position 49; I, Q, L, S, or A at position 50; D, S, A, or Q at position 101, W A, or N at position 103; Q, S, or A at position 105; Q, S or A at position 106; Q, S, or A at position 107; and Y or F at position 113; said positions determined with reference to SEQ ID NO:1; and (b) the VL region comprises: a CDR1 sequence, a CDR2, sequence, and a CDR3 sequence as set forth in Table 2 for the antibody designated L1A1; or at least one substitution in the CDR2 sequence, or CDR3 sequence, wherein the at least one substitution is selected from the group consisting of Q, S, or A, at position 49, Q, S, or A at position 50; and Y or F at position 85, said positions determined with reference to SEQ ID NO:2. In a further aspect, provided herein is an anti-HIV antibody comprising a heavy chain variable (VH) region and a light chain variable (VL) region, wherein: a) the VH region comprises a CDR1 sequence, a CDR2, sequence, and a CDR3 sequence as set forth in Table 2 for the antibody designated L1A1; or comprises at least one substitution in the CDR2 or CDR3, wherein the substitution is selected from the group consisting of Y or F at position 49; I, Q, L, S, or A at position 50; D, S, A, or Q at position 101, W A, or N at position 103; Q, S, or A at position 105; Q, S or A at position 106; Q, S, or A at position 107; and Y or F at position 113; said positions determined with reference to SEQ ID NO:1; and (b) the VL region comprises a CDR1 sequence, a CDR2, sequence, and a CDR3 sequence as set forth in Table 2 for the antibody designated L1A1 in which the CDR2 or CDR3 comprises at least one substitution, wherein the at least one substitution is selected from the group consisting of Q, S, or A, at position 49, Q, S, or A at position 50; and Y or F at position 85, said positions determined with reference to SEQ ID NO:2. In some embodiments, the VH region comprises at least one of the following, as numbered with reference to SEQ ID NO:1: V at position 1, Q at position 2, V at position 4, E at position 9, V at position 10, K at position 12, P at position 13, K at position 18, K at position 22, S at position 24, V at position 36, A at position 39, P at position 40, Q a position 42, M at position 47; R a position 66, I at position 75; S at position 76, M at position 80; E at position 81, L at position 82; S at position 83; R at position 84; R at position 86; S at position 87; L at position 123; V at position 124; or S at position 127; and/or the VL region comprises at least one of the following, as numbered with reference to SEQ ID NO:2: G at position 12; Y, A, V, L, or I at position 32; H at position 35; K at position 38; M at position 43, K at position 62, E at position 77; A at position 80; D at position 81, Y at position 83, and F at position 90. In some embodiments, the VH region has at least 70% identity, or at least 80% identity, or at least 90% identity to SEQ ID NO:1; and/or the VL region has at least 70% identity, or at least 80% identity, or at least 90% identity to SEQ ID NO:2.

In a further aspect, provided herein is an anti-HIV antibody comprising a heavy chain variable (VH) region and a light chain variable (VL) region, wherein: (a) the VH region comprises a CDR1 sequence, a CDR2, sequence, and a CDR3 sequence as set forth in Table 2 for the antibody designated L1A4 in which at least one of CDR2 or CDR3 comprises a substitution, wherein the substitution is selected from the group consisting of Y or F at position 49; I, Q, L, S, or A at position 50; E, S, or A at position 98; R or K at position 99; E, S, or A at position 101, A or S at position 102; Q, S, A, or G at position 104; Q, S, or A at position 105; E, S, or A at position 108; and Y or F at position 112; said positions determined with reference to SEQ ID NO:3; and (b) the VL region comprises: a CDR1 sequence, a CDR2, sequence, and a CDR3 sequence as set forth in Table 2 for the antibody designated L1A4; or at least one substitution in the CDR2 sequence, or CDR3 sequence, wherein the at least one substitution is selected from the group consisting of Q, S, or A, at position 49; Q, S, or A at position 50; and Y or F at position 85, said positions determined with reference to SEQ ID NO:4. In a further aspect, provided herein is an anti-HIV antibody comprising a heavy chain variable (VH) region and a light chain variable (VL) region, wherein: a) the VH region comprises a CDR1 sequence, a CDR2, sequence, and a CDR3 sequence as set forth in Table 2 for the antibody designated L1A4; or comprises at least one substitution in the CDR2 or CDR3, wherein the substitution is selected from the group consisting of Y or F at position 49; I, Q, L, S, or A at position 50; E, S, or A at position 98; R or K at position 99; E, S, or A at position 101, A or S at position 102; Q, S, A, or G at position 104; Q, S, or A at position 105; E, S, or A at position 108; and Y or F at position 112; said positions determined with reference to SEQ ID NO:3; and (b) the VL region comprises: a CDR1 sequence, a CDR2, sequence, and a CDR3 sequence as set forth in Table 2 for the antibody designated L1A4 comprising at least one substitution in the CDR2 sequence, or CDR3 sequence, wherein the at least one substitution is selected from the group consisting of Q, S, or A, at position 49; Q, S, or A at position 50; and Y or F at position 85, said positions determined with reference to SEQ ID NO:4. In some embodiments the VH comprises at least one of the following substitutions as numbered with reference to SEQ ID NO:3: V at position 1, Q at position 2, E at position 9, A at position 15, K position 22, S at position 24, V at position 36, T at position 68, T at position 73, S at position 74, I at position 75, S at position 76, Y at position 79, M at position 80, L at position 82, S at position 83, R at position 84, R at position 86, S at position 87, A at position 91, V at position 92, L at position 122, V at position 123, T at position 124, S at position 126, or S at position 127; and/or the VL comprises at least one of the follow substitutions as numbered with reference to SEQ ID NO:4: S or A at position 2; A at position 3; Y, A, V, L, or I at position 32; Q at position 34, K at position 38, M at position 43, I at position 44, V at position 54, K at position 62, A at position 76, Y at position 83, L at position 96, or T at position 97. In some embodiments, the VH comprises an amino acid sequence having at least 70% identity, or at least 80% identity, or at least 90% identity to SEQ ID NO:3; and/or the VL region comprises an amino acid sequence having at least 70% identity, or at least 80% identity, or at least 90% identity to SEQ ID NO:4.

In another aspect, provided herein is an anti-HIV antibody comprising a heavy chain variable (VH) region and a light chain variable (VL) region, wherein: (a) the VH region comprises a CDR1 sequence, a CDR2, sequence, and a CDR3 sequence as set forth in Table 2 for the antibody designated L2A1 in which at least one of CDR1 or CDR2 comprises a substitution, wherein the substitution is selected from the group consisting of N, R, Q, S, or A at position 27; Q, L, S or A at position 29; Y or F at position 49; I, Q, L, S, or A at position 50; Q, D, S or A at position 58; A or N at position 60; and Q, Y, or F at position 61; said positions determined with reference to SEQ ID NO:5; and (b) the VL region comprises: a CDR1 sequence, a CDR2, sequence, and a CDR3 sequence as set forth in Table 2 for the antibody designated L2A1; or at least one substitution in the CDR2 sequence, or CDR3 sequence, wherein the at least one substitution is selected from the group consisting of E, S, or A at position 46; E, S, or A at position 47; E, S, or A at position 48; Q, S, or A at position 49; Q, S, or A at position 50; and Q, S, A or W at position 85, said positions determined with reference to SEQ ID NO:6. In a further aspect, provided herein is an anti-HIV antibody that comprises a heavy chain variable (VH) region and a light chain variable (VL) region, wherein: a) the VH region comprises a CDR1 sequence, a CDR2, sequence, and a CDR3 sequence as set forth in Table 2 for the antibody designated L2A1; or comprises at least one substitution in the CDR1 or CDR2, wherein the substitution is selected from the group consisting of N, R, Q, S, or A at position 27; Q, L, S or A at position 29; Y or F at position 49; I, Q, L, S, or A at position 50; Q, D, S or A at position 58; A or N at position 60; and Q, Y, or F at position 61; said positions determined with reference to SEQ ID NO:5; and (b) the VL region comprises: a CDR1 sequence, a CDR2, sequence, and a CDR3 sequence as set forth in Table 2 for the antibody designated L2A1 in which the CDR2 sequence, or CDR3 sequence comprises at least one substitution, wherein the at least one substitution is selected from the group consisting of E, S, or A at position 46; E, S, or A at position 47; E, S, or A at position 48; Q, S, or A at position 49; Q, S, or A at position 50; and Q, S, A or W at position 85, said positions determined with reference to SEQ ID NO:6. In some embodiments, the VH comprises at least one of the following substitutions as numbered with reference to SEQ ID NO:5: A at position 8, E at position 9, P at position 13, S at position 16, K at position 18; V at position 19, K at position 22, A at position 23, S at position 24, T position 73, S at position 74, I at position 75, S at position 76, Y at position 79, S at position 83, R at position 84, S at position 87, T at position 121, L at position 122, or T at position 124; and/or the VL comprises at least one of the follow substitutions as numbered with reference to SEQ ID NO:6: Q at position 1, V at position 10, G at position 12, Y at position 32, H at position 35, A at position 39, M at position 43, Y at position 45, D at position 56, G at position 60, K at position 62, S at position 63, N at position 65, G at position 73, L at position 74, A at position 76, D at position 81, Y at position 83, or K at position 95. In some embodiments, the VH comprises an amino acid sequence having at least 70% identity, or at least 80% identity, or at least 90% identity to SEQ ID NO:5; and/or the VL region comprises an amino acid sequence having at least 70% identity, or at least 80% identity, or at least 90% identity to SEQ ID NO:6.

The invention additionally provided an expression vector comprising a polynucleotide encoding the VH region and/or the VL region of the anti-HIV antibody of any one of the preceding paragraphs in this section and a host cell that comprises such as expression vector; or a host cell that encodes the VH region and/or the VL region of the anti-HIV antibody.

In a further aspect, provided herein is a method of treating or preventing an HIV infection, the method comprising administering the anti-HIV antibody of any one of the preceding paragraphs in this section to a patient that is infected with an HIV virus, or is at risk of infection of with an HIV virus. In some embodiments, the method further comprises administering a latency reversing agent to the patient.

### BRIEF DESCRIPTION OF THE DRAWINGS

FIG. 1. Alignment of NVS49 antibodies L1A2, L1A1, L1A4, and L2A1. CDRs are indicated by shading in the numbered blocks. FIG. 1 discloses SEQ ID NOS 30-34, 1, 3, 5, 35-38, 2, 4, and 6, respectively, in order or appearance.

### DETAILED DESCRIPTION OF THE INVENTION

### Terminology

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

As used herein, the terms, “HIV antibody” and “anti-HIV antibody” are used synonymously to refer to an antibody that binds to an HIV antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the particular CDR is located. Thus, a V_{H} CDR3 (HCDR3) is located in the variable domain of the heavy chain of the antibody in which it is found, whereas a V_{L} CDR3 (LCDR3) is the CDR3 from the variable domain of the light chain of the antibody in which it is found. The term “CDR” is used interchangeably with “HVR” when referring to CDR sequences.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, Md. (1991)). Chothia CDRs are determined as defined by Chothia (see, e.g., Chothia and Lesk J. Mol. Biol. 196:901-917 (1987)).

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal, et al., Mol Immunol 30:105-108, 1993).

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹° M, less than 10⁻¹⁰ M, less than 5×10⁻¹ M, less than 10⁻¹¹ M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a bivalent antibody. In the context of the present invention, an “improved” K_{D} refers to a lower K_{D}. In some embodiments, an antibody of the present disclosure has a K_{D} of less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹⁰ M, less than 10⁻¹⁰ M, less than 5×10⁻¹¹ M, less than 10⁻¹¹M, less than 5×10⁻¹²M, less than 10⁻¹²M, less than 5×10⁻¹³ M, less than 10⁻¹³M, less than 5×10⁻¹⁴M, less than 10⁻¹⁴M, less than 5×10⁻¹⁵M, or less than 10⁻¹⁵ M or lower as measured as a monovalent antibody, such as a monovalent Fab. In some embodiments, an anti-HIV antibody of the present disclosure has K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, an anti-HIV antibody of the present disclosure has K_{D} of greater than 100 pM, e.g., in the range of 100-1000 pM or 500-1000 pM when measured by surface plasmon resonance analysis using a biosensor system such as a Biacore® system performed at 37° C.

The term “monovalent molecule” as used herein refers to a molecule that has one antigen-binding site, e.g., a Fab or scFv.

The term “bivalent molecule” as used herein refers to a molecule that has two antigen-binding sites. In some embodiments, a bivalent molecule of the present invention is a bivalent antibody or a bivalent fragment thereof. In some embodiments, a bivalent molecule of the present invention is a bivalent antibody. In some embodiments, a bivalent molecule of the present invention is an IgG. In general monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. Preferably both antigen-binding sites of a bivalent molecule share the same antigen specificity.

The term “valency” as used herein refers to the number of different binding sites of an antibody for an antigen. A monovalent antibody comprises one binding site for an antigen. A bivalent antibody comprises two binding sites for the same antigen.

The term “avidity” as used herein in the context of antibody binding to an antigen refers to the combined binding strength of multiple binding sites of the antibody. Thus, “bivalent avidity” refers to the combined strength of two binding sites.

The phrase “specifically (or selectively) binds” to an antigen or target or “specifically (or selectively) immunoreactive with,” when referring to a protein or peptide, refers to a binding reaction whereby the antibody binds to the antigen or target of interest. In the context of this invention, the antibody binds to HIV gp120.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a V_{H} region polypeptide “corresponds to” an amino acid in the V_{H} region of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cells and includes the primary transformed cell and progeny derived therefrom without regard to the number of passages.

A polypeptide “variant,” as the term is used herein, is a polypeptide that typically differs from a polypeptide specifically disclosed herein in one or more substitutions, deletions, additions and/or insertions. In the present invention, a “variant” with reference to the sequences described in the “Anti-HIV Antibody Variants” section refers to a engineered sequence, rather than a naturally occurring sequence. The term “sibling” as used herein with respect to an antibody refers to a naturally occurring antibody that exhibits similarity in aspects such as the same HV germline, same or similar H-CDR3 length, same LV germline, and same or similar L-CDR3 length; and may have arised from the same ancestral naïve B cell.

### Anti-HIV Antibody Variants

Provided herein are anti-HIV antibody variants of anti-HIV antibodies derived from a patient. In some embodiments, an anti-HIV antibody exhibits one or more improved properties compared to the naturally occurring counterpart from which it is derived. In some embodiments, a variant anti-HIV antibody as described herein has broadly neutralizing activity. In some embodiments, an anti-HIV antibody of the present disclosure comprises modifications compared to a naturally occurring antibody L1A1, L1A4, or L2A1 that provides improved pharmacokinetic properties, increased serum stability, increased binding affinity, and/or neutralization of HIV compared to the naturally occurring L1A1, L1A4, or L2A1 antibody from which the variant is derived. In some embodiments, a variant antibody as described herein exhibits reduced immunogenicity and/or increased efficiency of manufacture compared to the naturally occurring parent L1A1, L1Ar, or L2A1 antibody. In some embodiments, a variant anti-HIV antibody having at least one modification, e.g., substitution, relative to the native L1A1 variable or light chain sequence modification as described herein has improved development properties, e.g., decreased heterogeneity, increased yield, increased stability, improved net charges to improve pharmacokinetics, and or/reduced immunogenicity. In some embodiments, such an antibody has at least two, three, four, five, or six, or more modifications, e.g., substitutions, as described herein. In some embodiments, a variant anti-HIV antibody of the invention has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g., substitutions, in the variable region compared to L1A1. In some embodiments, a variant anti-HIV antibody having at least one modification, e.g., substitution, relative to the native L1A4 variable or light chain sequence modification as described herein has improved development properties, e.g., decreased heterogeneity, increased yield, increased stability, improved net charges to improve pharmacokinetics, and or/reduced immunogenicity. In some embodiments, such an antibody has at least two, three, four, five, or six, or more modifications, e.g., substitutions, as described herein. In some embodiments, a variant anti-HIV antibody of the invention a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g., substitutions, in the variable region compared to L1A4. In some embodiments, a variant anti-HIV antibody having at least one modification, e.g., substitution, relative to the native L2A1 variable or light chain sequence modification as described herein, has improved development properties, e.g., decreased heterogeneity, increased yield, increased stability, improved net charges to improve pharmacokinetics, and or/reduced immunogenicity. In some embodiments, such an antibody has at least two, three, four, five, or six, or more modifications, e.g., substitutions, as described herein. In some embodiments, a variant anti-HIV antibody of the invention has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g., substitutions, in the variable region compared to L2A1.

The variable region sequences of L1A1, L1A4, and L2A1 antibodies are provided in Table 1:

TABLE 1

| Name | VH amino acid sequence | VL amino acid sequence |
|---|---|---|
| NVS4 | AGLMQSGAVMKNSGASVRVSCQA | QSALTQPRSVSASPGQSVTISCTGTH |
| 9- | DGYDFIDYVIHWFRQRRGEGLEWL | NYVSWCQQKPGQAPKLLIYDFNKR |
| L1A1 | GWMNPSGGGTNYPRPFQGKVTMT | PSGVSDRFSGSTSGNTASLTISGLQA |
| | RDTSTETAYLDVRGLTYDDTAVYY | DDEGHYFCWAFENIGGGTKLTVL |
| | CVRDRANGSGRRRFESVNWFLDLW | (SEQ ID NO: 2) |
| | GRGTQITVVS (SEQ ID NO: 1) | |
| NVS4 | SAELVQSGAVVKKPGTSVKVSCQA | QCVLTQPRSVSGSPGQSVTISCTGT |
| 9- | YGYTFTDYLIHWLRQAPGQGLEWM | HNYVSWCQHHPGNAPKLLLYDFD |
| L1A4 | GWMNPVYGQVNYAQNFQGRVSM | KRPSGISDRFSGSRSGNTASLTISGL |
| | TRDIYRETAFLEVRDLKTDDTGTYY | QPEDEADYFCWAFEAFGGGTKVLV |
| | CVRDTGDGSRRHFDSINWFLDLWG | L (SEQ ID NO: 4) |
| | RGTWIRVAP (SEQ ID NO: 3) | |
| NVS4 | HVQLVQSGGGVKKIGAAVRISCEVT | ASALTQPASMSASPGQSVTISCSGT |
| 9- | GYKFMDQLINWVRQAPGQGLEWM | RHIISAWFQQYPGKPPKLIIFDDDKR |
| L2A1 | GWMNPTYGQVNYSWRFEGRVTMT | PSGVPSRFSASRPGDTASLTISNVQP |
| | RDMDTETAFMELRGLRVDDTAVY | EDEATYICNTYEFFGGGTRLTVL |
| | YCARGPSGENYPFHYWGQGVRVV | (SEQ ID NO: 6) |
| | VSS (SEQ ID NO: 5) | |

The heavy and light chain CDRs of L1A1, L1A4, and L2A1 are shown in Table 2:

| | CDR1 | CDR2 | CDR3 |
|---|---|---|---|
| L1A1 VH | GYDFIDYVIH | WMNPSGGGTNYPRPFQG | VRDRANGSGRRRFESVNWFLDL |
| region | (SEQ ID NO: 13) | (SEQ ID NO: 14) | (SEQ ID NO: 15) |
| L1A1 VL | TGTHNYVS | DFNKRPS | WAFEN |
| region | (SEQ ID NO: 16) | (SEQ ID NO: 17) | (SEQ ID NO: 18) |
| L1A4 VH | GYTFTDYLIH | WMNPVYGQVNYAQNFQG | VRDTGDGSRRHFDSINWFLDL |
| region | (SEQ ID NO: 19) | (SEQ ID NO: 20) | (SEQ ID NO: 21) |
| L1A4 VL | TGTHNYVS | DFDKRPS | WAFEA |
| region | (SEQ ID NO: 16) | (SEQ ID NO: 22) | (SEQ ID NO: 23) |
| L2A1 VH | GYKFMDQLIN | WMNPTYGQVNYSWRFEG | ARGPSGENYPFHY |
| region | (SEQ ID NO: 24) | (SEQ ID NO: 25) | (SEQ ID NO: 26) |
| L2A1 VL | SGTRHIISA | DDDKRPS | NTYEF |
| region | (SEQ ID NO: 27) | (SEQ ID NO: 28) | (SEQ ID NO: 29) |

Position 127 of SEQ ID NO:1 and position 99 of SEQ ID NO:2 are considered to be the last amino acids of the V_{H} and V_{L} regions, respectively, according to EU index numbering. In a human IgG format (e.g., IgG1, IgG2, IgG3, or IgG4), the subsequent residue is termed the “junction codon”, and is natively encoded by the junction of the final 3′ base of the variable region gene (HJ or LJ) with the first two 5′ bases of the constant region gene (heavy or light), and exhibits amino acid variation due to variation in the final 3′ base of HJ and LJ. The human heavy chain junction codon can natively be Ala, Ser, Pro, or Thr, and is usually an Ala. The human kappa chain junction codon can natively be Arg or Gly, and is usually an Arg. The human lambda chain junction codon can natively be Gly, Ser, Arg, or Cys, and is usually a Ser or Gly.

### L1A1 Variants

In some embodiments, an anti-HIV antibody of the present invention has one, two, or three CDRs of a V_{H} sequence of the antibody designated as L1 Al in Table 1; and at least one mutation in the amino acid sequence of the L1A1 antibody as shown in Table 1. The at least one mutation may be in the CDRs or framework regions. In some embodiments, the V_{H} CDR2 or CDR3 sequence has 1, 2, 3, 4, or 5 substitutions relative to the L1A1 CDR2 or CDR3 sequence shown in Table 2. In some embodiments, the V_{H} region comprises an L1A1 CDR1 as shown in Table 2; or has 1, 2, 3, or 4 substitutions, e.g., conservative substitutions.

In some embodiments, an anti-HIV antibody of the present invention has a VH that comprises a L1A1 CDR2 sequence as shown in Table 2 in which one or two positions 49 and 50, as numbered with reference to SEQ ID NO:1, are substituted. In some embodiments, the V_{H} region comprises the CDR2 sequence shown Table 2 in which position 49 is Y or F; and/or position 50 is I, Q, L, S, or A. In some embodiments, the CDR2 comprises one, two, or three additional substitutions, e.g., conservative substitutions. In some embodiments, the CDR2 has at least 70% or at least 75% identity, or at least 80% identity, to the L1A2 CDR2 sequence shown in Table 2.

In some embodiments, an anti-HIV antibody of the present invention has a V_{H} that comprises a L1A1 CDR3 sequence as shown in Table 2 in which one or two of positions 101, 103, 105, 106, 107, or 113, as numbered with reference to SEQ ID NO:1, is substituted; or in which three, four, five, or all six positions are substituted. In some embodiments, the V_{H} region comprises the CDR3 sequence shown Table 2 in which a position 101, 103, 105, 106, 107, or 113, as numbered with reference to SEQ ID NO:1, is substituted and the substitution is selected from the group consisting of D, A, S, or Q at position 101; W, A, or N at position 103; Q, S, or A at positions 105; Q, S, or A at position 106; Q, S, or A at position 107; and Y or F at position 113. In some embodiments, the CDR3 comprises a substitution at position 101, 103, 105, 106, 107, or 113 as designated in the preceding sentence and at least 1, 2, 3, or 4 additional substitutions in the CDR2 sequence. In some embodiments, the CDR3 comprises substitutions at least two or three of positions 101, 103, 105, 106, 107, or 113, wherein the substitutions are selected from the group consisting of D, A, S, or Q at position 101; W, A, or N at position 103; Q, S, or A at positions 105; Q, S, or A at position 106; Q, S, or A at position 107; and Y or F at position 113. In some embodiments, the CDR3 comprises substitutions at four, five, or all of positions 101, 103, 105, 106, 107, or 113, wherein the substitution is selected from the group consisting of D, A, S, or Q at position 101; W, A, or N at position 103; Q, S, or A at positions 105; Q, S, or A at position 106; Q, S, or A at position 107; and Y or F at position 113. In some embodiments, the substitution is at position 113. In some embodiments, the substitutions is at position 105, 106, or 107. In some embodiments, the substitution is at position 101. In some embodiments, the substitution is at position 103. In some embodiments, the CDR3 has at least 70% identity, at least 75% or at least 80% identity to the L1A1 CDR3 sequence set forth in Table 2 and comprises at one or more substitutions at position 101, 103, 105, 106, 107, or 113; wherein the substitution is selected from the group consisting of D, A, S, or Q at position 101; W, A, or N at position 103; Q, S, or A at positions 105; Q, S, or A at position 106; Q, S, or A at position 107; and Y or F at position 113. In some embodiments, the CDR3 comprises one, two, or three additional substitutions, e.g., conservative substitutions.

In some embodiments, an anti-HIV antibody of the present invention comprises a V_{H} region CDR2 and/or a CDR3 as described in the previous two paragraphs and has at least 70%, at least 75%, at least 80% identity, at least 85% identity, at least 90% identity, or at least 95% identity to SEQ ID NO:1. In some embodiments, the antibody further comprises at least one of the following, as numbered with reference to SEQ ID NO:1: V at position 1, Q at position 2, V at position 4, E at position 9, V at position 10, K at position 12, P at position 13, K at position 18, K at position 22, S at position 24, V at position 36, A at position 39, P at position 40, Q at position 42, M at position 47, R at position 66, I at positions 75, S at position 76, M at position 80, E at position 81, L at position 82, S at position 83, R at position 84, R at position 86, Y at position 87, L at position 123, V at position 124, and S at position 127. In some embodiments, the V_{H} region includes an additional amino acid at the N-terminal end (position “0”), e.g., Q.

In some embodiments, an anti-HIV antibody of the present invention comprises a V_{H} region having at least 70%, at least 75%, at least 80% identity, at least 85% identity, at least 90% identity, or at least 95% identity to SEQ ID NO:1, wherein the variant comprises Y or F at position 49; I, Q, L, S, or A at position 50; D, A, S, or Q at position 101; W, A, or N at position 103; Q, S, or A at positions 105; Q, S, or A at position 106; Q, S, or A at position 107; Y or F at position 113; V at position 1, Q at position 2, V at position 4, E at position 9, V at position 10, K at position 12, P at position 13, K at position 18, K at position 22, S at position 24, V at position 36, A at position 39, P at position 40, Q at position 42, M at position 47, R at position 66, I at positions 75, S at position 76, M at position 80, E at position 81, L at position 82, S at position 83, R at position 84, R at position 86, Y at position 87, L at position 123, V at position 124, or S at position 127 as numbered with reference to SEQ ID NO:1. In some embodiments, the V_{H} region includes an additional amino acid at the N-terminal end (position “0”), e.g., Q.

In some embodiments, an anti-HIV antibody comprises a L1A1 CDR2 and/or a CDR3 as described in the preceding paragraphs in this section and a L1A1 CDR1 as shown in Table 2 or a CDR1 having 1, 2, or 3 substitutions, e.g., conservative substitutions, relative to the L1A2 CDR1 of Table 2 and comprises two, three, four, or five additional amino acid changes relative to SEQ ID NO:1, but no more than thirty, or thirty-five, additional changes. In some embodiments, the antibody comprises at least six, seven, eight, nine or ten additional amino changes relative to SEQ ID NO:1, but no more than, but no more than thirty, or thrity-five, additional changes. In some embodiments are selected from those described in the preceding paragraph.

In some embodiments, an anti-HIV antibody of the present invention has one, two, or three CDRs of a V_{L} sequence of the antibody designated as L1A1 in Table 1; and at least one mutation, e.g., a deletion, substitution, or addition, in the amino acid sequence of the L1A1 V_{L} region as shown in Table 1. In some embodiments, the CDR1, CDR2 or CDR3 sequence comprises a substitution relative to the V_{L} region CDR1, CDR2, or CDR3 L1A1 sequences shown in Table 2.

In some embodiments, an anti-HIV antibody of the present invention has a V_{L} that comprises a L1A1 CDR2 sequence as shown in Table 2 in which position 49 is substituted with Q, S, or A; and/or position 50 is substituted with Q, S, or A. In some embodiments, the CDR2 comprises 1 or 2 additional substitutions, e.g., conservative substitutions, relative to the CDR2 sequence as shown in Table 2. In some embodiments, an anti-HIV antibody of the present disclosure comprises a V_{L} region comprising a CDR3 sequence as shown in Table 2 in which position 85 is substituted with F. In some embodiments, the CDR3 comprises 1 or 2 additional substitutions relative to the sequence shown in Table 2.

In some embodiments, an anti-HIV antibody of the present invention comprises a V_{L} region CDR1, CDR2, and/or a CDR3 as described in the previous paragraphs in this section and is at least 70% identity, at least 75%, at least 80%, at least 85% identity, at least 90% identity, or at least 95% identity to SEQ ID NO:2. In some embodiments, an anti-HIV antibody having substitution in a V_{L} CDR1, CDR2, and/or CDR3 further comprises at least one of the following, as numbered with reference to SEQ ID NO:2: G at position 12; Y, A, V, L, or I at position 32, H at position 35, K at position 38, M at position 43, K at position 62, E at position 77, A at position 80, D at position 81, Y at position 83, or F at position 90.

In some embodiments, an anti-HIV antibody comprises a CDR1, CDR2 and/or a CDR3 as described in this section and comprises two, three, four, or five additional amino acid changes relative to SEQ ID NO:2, but no more than thirty, or no more than thirty-five additional changes. In some embodiments, the antibody comprises at least six, seven, eight, nine or ten additional amino changes relative to SEQ ID NO:2, but no more than thirty, or no more than thirty-five, additional changes.

In some embodiments, an anti-HIV antibody of the present invention comprises a VL region having at least 70% identity, at least 75%, at least 80%, at least 85% identity, at least 90% identity, or at least 95% identity to SEQ ID NO:2, and comprises at least one of the following: Q, S, or A at position 49; Q, S, or A at position 50; F at position 85; G at position 12; Y, A, V, L, or I at position 32, H at position 35, K at position 38, M at position 43, K at position 62, E at position 77, A at position 80, D at position 81, Y at position 83, or F at position 90.

In some embodiments, an anti-HIV antibody of the present invention comprises an L1A1 variant comprising a V_{H} region as described in the preceding paragraphs and a VL region as described in the preceding paragraphs in this section.

### L1A4 Variants

In some embodiments, an anti-HIV antibody of the present invention has one, two, or three CDRs of a V_{H} sequence of the antibody designated as L1A4 in Table 1; and at least one mutation in the L1A4 amino acid sequence oas shown in Table 1. The at least one mutation may be in the CDRs or in framework regions. In some embodiments, the V_{H} CDR2 or CDR3 sequence has 1, 2, 3, 4, or 5 substitutions relative to the L1A4 CDR2 or CDR3 sequence shown in Table 2. In some embodiments, the V_{H} region comprises an L1A4 CDR1 as shown in Table 2; or has 1, 2, 3, or 4 substitutions, e.g., conservative substitutions. In some embodiments, the CDR2 or CDR3 sequence has 1, 2, 3, 4, or five substitutions relative to the L1A4 CDR2 or CDR3 sequence shown in Table 2.

In some embodiments, an anti-HIV antibody of the present invention has a V_{H} that comprises a L1A4 CDR2 sequence as shown in Table 2 in which position 49 and/or position 50, as numbered with reference to SEQ ID NO:3, are substituted. In some embodiments, the V_{H} region comprises the L1A4 CDR2 sequence shown Table 2 in which position 49 is Y or F; and/or position 50 is I, Q, L, S, or A. In some embodiments, the CDR2 has at least 70% or at least 75% identity, or at least 80% identity, to the L1A4 CDR2 sequence shown in Table 2.

In some embodiments, an anti-HIV antibody of the present invention has a V_{H} that comprises a L1A4 CDR3 sequence as shown in Table 2 in which one or two positions 98, 99, 101, 102, 104, 105, 108, or 112, as numbered with reference to SEQ ID NO:3, is substituted; or in which three, four, or five of the positions are substituted; or in which six, seven, or all of the positions are substituted. In some embodiments, the V_{H} region comprises the L1A4 CDR3 sequence shown Table 2 in which one or more of positions 98, 99, 101, 102, 104, 105, 108, or 112, as numbered with reference to SEQ ID NO:1, is substituted and the substitution is selected from the group consisting of E, S, or A at position 98; R or K at position 99; E, S, or A at position 101; A or S at position 102; Q, S, A, or G at position 104; Q, S, or A at position 105, E, S, or A at position 108; and Y or F at position 112. In some embodiments, the CDR3 comprises one substitution at position 98, 99, 101, 102, 104, 105, 108, or 112 as designated in the preceding sentence. In some embodiments, the CDR3 comprises substitutions at two, or three, of positions 98, 99, 101, 102, 104, 105, 108, or 112, wherein the substitution is selected from the group consisting of E, S, or A at position 98; R or K at position 99; E, S, or A at position 101; A or S at position 102; Q, S, A, or G at position 104; Q, S, or A at position 105, E, S, or A at position 108; and Y or F at position 112. In some embodiments, the CDR3 comprises substitutions at four, at five, at six, at seven, or all of positions 101, 103, 105, 106, 107, or 113, wherein the substitution is selected from the group consisting of E, S, or A at position 98; R or K at position 99; E, S, or A at position 101; A or S at position 102; Q, S, A, or G at position 104; Q, S, or A at position 105, E, S, or A at position 108; and Y or F at position 112. In some embodiments, the CDR3 has at least 80% identity to the L1A4 CDR3 sequence set forth in Table 2 and comprises at least one substitution at position 98, 99, 101, 102, 104, 105, 108, or 112; wherein the substitution is selected from the group consisting of E, S, or A at position 98; R or K at position 99; E, S, or A at position 101; A or S at position 102; Q, S, A, or G at position 104; Q, S, or A at position 105, E, S, or A at position 108; and Y or F at position 112. In some embodiments, the CDR3 has at least 70% or at least 75% identity, or at least 80% identity, to the L1A4 CDR3 sequence shown in Table 2.

In some embodiments, an anti-HIV antibody of the present invention comprises a V_{H} region L1A4 CDR2 and/or a CDR3 as described in the preceding paragraphs in this section and has at least 70%, at least 75%, at least 80% identity, at least 85% identity, at least 90% identity, or at least 95% identity to SEQ ID NO:3. In some embodiments, the antibody further comprises at least one of the following, as numbered with reference to SEQ ID NO:3: V at position 1, Q at position 2, E at position 9, A at position 15, K at position 22, S at position 24, V at position 36, T at position 68, T at position 73, Y at position 74, I at position 75, S at position 76, Y at position 79, M at position 80, L at position 82, S at position 83, R at position 84, R at position 86, S at position 87, A at position 91, V at position 92, L at position 122, V at position 123, T at position 124, S at position 126, or S at position 127. In some embodiments, the V_{H} region includes an additional amino acid at the N-terminal end (position “0”), e.g., Q.

In some embodiments, an anti-HIV antibody of the present invention comprises a V_{H} region having at least 70%, at least 75%, at least 80% identity, at least 85% identity, at least 90% identity, or at least 95% identity to SEQ ID NO:3, wherein the V_{H} region comprises at least one of the following: Y or F at position 49; I, Q, L, S, or A at position 50; E, S, or A at position 98; R or K at position 99; E, S, or A at position 101; A or S at position 102; Q, S, A, or G at position 104; Q, S, or A at position 105, E, S, or A at position 108; Y or F at position 112, V at position 1, Q at position 2, E at position 9, A at position 15, K at position 22, S at position 24, V at position 36, T at position 68, T at position 73, Y at position 74, I at position 75, S at position 76, Y at position 79, M at position 80, L at position 82, S at position 83, R at position 84, R at position 86, S at position 87, A at position 91, V at position 92, L at position 122, V at position 123, T at position 124, S at position 126, or S at position 127 as numbered with reference to SEQ ID NO:3. In some embodiments, the V_{H} region includes an additional amino acid at the N-terminal end (position “0”), e.g., Q.

In some embodiments, an anti-HIV antibody comprises a CDR2 and/or a CDR3 as described in the preceding paragraphs in this section and comprises two, three, four, or five additional amino acid changes relative to SEQ ID NO:3, but no more than thirty, or no more than thirty-five, additional changes. In some embodiments, the antibody comprises at least six, seven, eight, nine or ten additional amino changes relative to SEQ ID NO:3, but no more than thirty, or no more than thirty-five, additional changes. In some embodiments are selected from those described in the preceding paragraph.

In some embodiments, an anti-HIV antibody of the present invention has at one, two, or three CDRs of a V_{L} sequence of the antibody designated as L1A4 in Table 1; and at least one mutation, e.g., a deletion, substitution, or addition, in the L1A4 amino acid sequence as shown in Table 1. In some embodiments, the CDR1, CDR2 or CDR3 sequence comprises a substitution relative to the L1A4 V_{L} region CDR1, CDR2, or CDR3 sequence shown in Table 2.

In some embodiments, an anti-HIV antibody of the present invention has a V_{L} that comprises an L1A4 CDR2 sequence as shown in Table 2 in which position 49 is substituted with Q, S, or A; and/or position 50 is substituted with Q, S, or A. In some embodiments, the CDR2 comprises 1 or 2 additional substitutions, e.g., conservative substitutions, relative to the CDR2 sequence as shown in Table 2. In some embodiments, an anti-HIV antibody of the present disclosure comprises a V_{L} region comprising an L1A4 CDR3 sequence as shown in Table 2 in which position 85 is substituted with F or Y. In some embodiments, the CDR3 comprises 1 or 2 additional substitutions relative to the sequence shown in Table 2.

In some embodiments, an anti-HIV antibody of the present invention comprises a V_{L} region CDR1, CDR2, and/or a CDR3 as described in the preceeding paragraphs in this section and is at least 70% identity, at least 75%, at least 80%, at least 85% identity, at least 90% identity, or at least 95% identity to SEQ ID NO:4. In some embodiments, an anti-HIV antibody having substitution in a V_{L} CDR1, CDR2, and/or CDR3 further comprises at least one of the following, as numbered with reference to SEQ ID NO:4: S or A at position 3; A at position 3; Y, A, V, L, or I at position 32; Q at position 34; K at position 38; M at position 43; I at position 44V at position 54, K at position 62, A at position 76, Y at position 83, L at position 96, or T at position 97.

In some embodiments, an anti-HIV antibody comprises a CDR1, CDR2 and/or a CDR3 as described in the preceding paragraphs in this section and comprises two, three, four, or five additional amino acid changes relative to SEQ ID NO:4, but no more than thirty, or no more than thirty-five, additional changes. In some embodiments, the antibody comprises at least six, seven, eight, nine or ten additional amino changes relative to SEQ ID NO:4, but no more than, but no more than thirty, or no more than thirty-five, additional changes.

In some embodiments, an anti-HIV antibody of the present invention comprises a V_{L} region having at least 70% identity, at least 75%, at least 80%, at least 85% identity, at least 90% identity, or at least 95% identity to SEQ ID NO:4, and comprises at least one of the following: Q, S, or A at position 49; Q, S, or A at position 50; F or Y at position 85; S or A at position 3; A at position 3; Y, A, V, L, or I at position 32; Q at position 34; K at position 38; M at position 43; I at position 44V at position 54, K at position 62, A at position 76, Y at position 83, L at position 96, or T at position 97 as determined with reference to SEQ ID NO:4.

In some embodiments, an anti-HIV antibody of the present invention comprises a V_{H} region as described in the preceding paragraphs and a V_{L} region as described in the preceding paragraphs.

### L2A1 Variants

In some embodiments, an anti-HIV antibody of the present invention has at one, two, or three CDRs of a V_{H} sequence of the antibody designated as L2A1 in Table 1; and at least one mutation in the amino acid sequence of the L2A1 antibody shown in Table 1. The at least one mutation may be in the CDRs or in framework regions. In some embodiments, the V_{H} CDR1 or CDR2 sequence has 1, 2, 3, 4, or 5 substitutions relative to the L2A1 CDR1 or CDR2 sequence shown in Table 2. In some embodiments, the V_{H} region comprises an L2A1 CDR3 as shown in Table 2 that has 1 or 2 substitutions.

In some embodiments, an anti-HIV antibody of the present invention has a V_{H} that comprises a L2A1 CDR1 sequence as shown in Table 2 in which position 27 and/or position 29, as numbered with reference to SEQ ID NO:5, are substituted. In some embodiments, the V_{H} region comprises the CDR1 sequence shown Table 2 in which position 27 is N, R, Q, S, or A; and/or position 29 is Q, S, L, or A.

In some embodiments, an anti-HIV antibody of the present invention has a V_{H} that comprises a L2A1 CDR2 sequence as shown in Table 2 in which one or two of positions 49, 50, 58, 60, or 61, as numbered with reference to SEQ ID NO:5, are substituted; or in which three, four, or all of the positions are substituted. In some embodiments, the V_{H} region comprises the L2A1 CDR2 sequence shown Table 2 in which one or more of positions 49, 50, 58, 60, or 61, as numbered with reference to SEQ ID NO:5 is substituted and the substitution is selected from the group consisting of Y or F at position 49; I, Q, L, S, or A at position 50; Q, D, S, or A at position 58, A or N at position 60; and Y or F at position 61. In some embodiments, the V_{H} region comprises the L2A1 CDR2 sequence shown Table 2 in which two or three of positions 49, 50, 58, 60, or 61, as numbered with reference to SEQ ID NO:5 is substituted and the substitutions are selected from the group consisting of Y or F at position 49; I, Q, L, S, or A at position 50; Q, D, S, or A at position 58, A or N at position 60; and Y or F at position 61. In some embodiments, the V_{H} region comprises the L2A1 CDR2 sequence shown Table 2 in which four or five of positions 49, 50, 58, 60, or 61, as numbered with reference to SEQ ID NO:5 is substituted and the substitutions are selected from the group consisting of Y or F at position 49; I, Q, L, S, or A at position 50; Q, D, S, or A at position 58, A or N at position 60; and Y or F at position 61. In some embodiments, the CDR2 has at least 70% or at least 75% identity, or at least 80% identity, to the L2A1 CDR2 sequence shown in Table 2. In some embodiments, the V_{H} region comprises a CDR2 as described in this paragraph and a CDR1 as described in the preceding paragraph. In some embodiments, the antibody comprises a CDR1 and/or CDR2 as described in this paragraph and the preceding paragraph; and an L2A1 CDR3 as shown in Table 2; or an L2A1 CDR3 as shown in Table 2 having at 1, 2, 3, 4, 5, or 6 mutations relative to the CDR3 sequence as shown in Table 2. In some embodiments, the CDR3 has at least 70% or at least 75% identity, or at least 80% identity, to the L2A1 CDR3 sequence shown in Table 2.

In some embodiments, an anti-HIV antibody of the present invention comprises a V_{H} region CDR1 and/or a CDR2 as described in the preceding paragraphs is this section and is at least 70%, at least 75%, at least 80% identity, at least 85% identity, at least 90% identity, or at least 95% identity to SEQ ID NO:5. In some embodiments, the antibody further comprises at least one of the following, as numbered with reference to SEQ ID NO:5: A at position 8, E at position 9, P at position 13, S at position 16; K at position 18, V at position 19, K at position 22, A at position 23, S at position 24, T at position 73, S at position 74, I at position 75, S at position 76, Y at position 79, S at position 83, R at position 84, S at position 87, T at position 121, I at position 122, or T at position 124. In some embodiments, the V_{H} region includes an additional amino acid at the N-terminal end (position “0”), e.g., Q.

In some embodiments, provided herein is an anti-HIV antibody comprising the CDR1, CDR2, and CDR3 of a V_{H} region of any one of SEQ ID NO:7, SEQ ID NO:8, SEQ ID NO:9, SEQ ID NO:10, SEQ ID NO:11, or SEQ ID NO:12.

In some embodiments, an anti-HIV antibody of the present invention comprises a V_{H} region having is at least 70%, at least 75%, at least 80% identity, at least 85% identity, at least 90% identity, or at least 95% identity to SEQ ID NO:5; and comprising at least one of the following, as determined with reference to SEQ ID NO:5 N, R, Q, S, or A at position 27; Q, S, L, or A at position 29; Y or F at position 49; I, Q, L, S, or A at position 50; Q, D, S, or A at position 58; A or N at position 60; Y or F at position 61; A at position 8, E at position 9, P at position 13, S at position 16; K at position 18, V at position 19, K at position 22, A at position 23, S at position 24, T at position 73, S at position 74, I at position 75, S at position 76, Y at position 79, S at position 83, R at position 84, S at position 87, T at position 121, I at position 122, or T at position 124. In some embodiments, the V_{H} region includes an additional amino acid at the N-terminal end (position “0”), e.g., Q.

In some embodiments, an anti-HIV antibody comprises a V_{H} CDR1 and/or a CDR2 as described in the previous three paragraphs and comprises two, three, four, or five additional amino acid changes relative to SEQ ID NO:5, but no more than thirty, or no more then thirty-five, additional changes. In some embodiments, the antibody comprises at least six, seven, eight, nine or ten additional amino changes relative to SEQ ID NO:5, but no more than thirty, or no more then thirty-five, additional changes. In some embodiments are selected from those described in the preceding paragraph.

In some embodiments, an anti-HIV antibody of the present invention has one, two, or three CDRs of a V_{L} sequence of the antibody designated as L2A1 in Table 1; and at least one mutation, e.g., a deletion, substitution, or addition, in the amino acid sequence of L2A1 as shown in Table 1. In some embodiments, the CDR1, CDR2 or CDR3 sequence comprises a substitution relative to the L2A1 V_{L} region CDR1, CDR2, or CDR3 sequence shown in

Table 2.

In some embodiments, an anti-HIV antibody of the present invention has a V_{L} that comprises an L2A1 CDR2 sequence as shown in Table 2 in which one position, or in some embodiments two positions, 46, 47, 48, 49, and 50, as numbered with reference to SEQ ID NO:6, are substituted; or in which three, four, or all five of the positions are substituted. In some embodiments, the V_{L} region comprises the L2A1 CDR2 sequence shown Table 2 in which one or more of positions 46, 47, 48, 49, and 50, as numbered with reference to SEQ ID NO:6 is substituted and the substitution is selected from the group consisting of E, S, or A at position 46; E, S, or A at position 47; E, S, or A at position 48; Q, S or A at position 49; and Q, S, or A at position 50. In some embodiments, the V_{H} region comprises the CDR2 sequence shown Table 2 in which two or three of positions 46, 47, 48, 49 or 50, as numbered with reference to SEQ ID NO:6 is substituted and the substitution is selected from the group consisting of E, S, or A at position 46; E, S, or A at position 47, E, S, or A at position 48; Q, S or A at position 49; and Q, S, or A at position 50. In some embodiments, the V_{H} region comprises the L2A1 CDR2 sequence shown Table 2 in which four or five positions 46, 47, 48, 49 or 50, as numbered with reference to SEQ ID NO:6, are substituted and the substitution is selected from the group consisting of E, S, or A at position 46; E, S, or A at position 47, E, S, or A at position 48; Q, S or A at position 49; and Q, S, or A at position 50.

In some embodiments, an anti-HIV antibody of the present invention comprises an L2A1 V_{L} CDR3 of Table 2 having a substitution at position 85, as determined with reference to SEQ ID NO:6. In some embodiments, position 85 is a Q, S, A, or W. In some embodiments, the V_{H} region comprises a CDR3 as described in this paragraph, a CDR2 as set forth in Table 2 or in the preceding paragraph and a CDR1 as set forth in Table 2. In some embodiments, the antibody has 1, 2, or 3 substitutions, e.g., conservative substitutions, in the CDR1 relative to the sequence set forth in Table 2.

In some embodiments, an anti-HIV antibody of the present invention comprises a V_{L} region CDR1, CDR2, and/or a CDR3 as described in the preceding paragraphs in this section and has at least 70% identity, at least 75%, at least 80%, at least 85% identity, at least 90% identity, or at least 95% identity to SEQ ID NO:6. In some embodiments, the V_{L} region of the antibody further comprises at least one of the following, as numbered with reference to SEQ ID NO:6: Q at position 1, V at position 10, G at position 12, Y at position 32, H at position 35, A at position 39, M at position 43, Y at position 45, or K at position 95.

In some embodiments, an anti-HIV antibody comprises a CDR1, CDR2 and/or a CDR3 as described in the preceding paragraphs in this section and comprises two, three, four, or five additional amino acid changes relative to SEQ ID NO:6, but no more than thirty, or no more then thirty-five, additional changes. In some embodiments, the antibody comprises at least six, seven, eight, nine or ten additional amino changes relative to SEQ ID NO:6, but no more than thirty, or no more then thirty-five, additional changes.

In some embodiments, an anti-HIV antibody of the present invention comprises a V_{L} region having at least 70% identity, at least 75%, at least 80%, at least 85% identity, at least 90% identity, or at least 95% identity to SEQ ID NO:6, and comprises at least one of the following: E, S, or A at position 46; E, S, or A at position 47, E, S, or A at position 48; Q, S or A at position 49; and Q, S, or A at position 50; Q, S, A, or W at position 85; Q at position 1, V at position 10, G at position 12, Y at position 32, H at position 35, A at position 39, M at position 43, Y at position 45, or K at position 95.

In some embodiments, an anti-HIV antibody of the present invention comprises a V_{H} region as described in the preceding paragraphs and a V_{L} region as described in the preceding paragraphs in this section.

### Antibody Formats

In a further aspect of the invention, an anti-HIV antibody according to any of the above embodiments is a monoclonal antibody, including a chimeric, antibody. In one embodiment, an anti-HIV antibody is an antibody fragment, e.g., a Fv, Fab, Fab′, scFv, diabody, or F(ab′)₂ fragment. In another embodiment, the antibody is a substantially full length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells (e.g. E. coli or phage), as described herein.

In some embodiments an anti-HIV antibody in accordance with the present disclosure is a in a monovalent format. In some embodiments, the anti-HIV antibody is in a fragment format, e.g., a Fv, Fab, Fab′, scFv, diabody, or F(ab′)₂ fragment.

In some embodiments, an anti-HIV antibody of the present invention is employed in a bispecific or multi-specific format. For example, in some embodiments, the antibody may be incorporated into a bispecific or multi-specific antibody that comprises a further binding domain that binds to the same or a different antigen.

In some embodiments, an antibody of the present disclosure comprises an Fc region. The Fc region may be an Fc region engineered to alter one or more functional properties of the antibody, such as serum half-life, complement fixation, Fc receptor binding, and/or antigen-dependent cellular cytotoxicity. Furthermore, an antibody of the disclosure may be chemically modified (e.g., one or more chemical moieties can be attached to the antibody) or be modified to alter its glycosylation, again to alter one or more functional properties of the antibody. Additional modifications may also be introduced. For example, the antibody can be linked to one of a variety of polymers, for example, polyethylene glycol.

### Activity

The activity of an anti-HIV antibody variant as described herein can be assessed for binding to HIV, neutralization potency, and/or neutralization breadth. In some embodiments, effector function, e.g., ADCC, is also evaluated.

A “neutralizing anti-HIV antibody” as used herein refers to an antibody that can prevent HIV from initiating and perpetuating an infection in a host and/or in target cells in vitro. In some embodiments, the present invention provides neutralizing monoclonal human antibodies that bind to HIV gp120 polypeptide.

As used herein, “broadly neutralizing antibodies” refers to antibodies that neutralize multiple HIV-1 virus strains from diverse clades and different strains within a Glade in a neutralization assay. In some embodiments, a broadly neutralizing antibody may neutralize at least 50 or more different strains of HIV-1. In certain embodiments, the 50% inhibitory concentration of the monoclonal antibody may be less than about 0.0001 μg/ml, less than about 0.001 μg/ml, less than about 0.01 μg/ml, less than about 1 μg/ml, less than about 5 μg/ml, less than about 10 μg/ml, less than about 20 μg/ml, less than about 50 μg/ml, or less than about 100 μg/ml and is defined as the antibody concentration required to neutralize about 50% of the input virus in the neutralization assay.

Broadly neutralizing activity of an antibody can be determined by evaluating neutralization against a panel of HIV-1 viruses, which in some embodiments, includes viruses from multiple clades and circulating recombinant forms. These can include both chronic as well as transmitted/founder (T/F) viruses. Such assays can be performed using panels of appropriate HIV-1 pseudoviruses using methodology such as that described by Decamp et al., J. Virol. 88:2489-2507, 2014, Seaman et al., J Virol. 54: 1439-1452, 2010; or Hraber et al., J. Virol. 91: e00991-17, 2017. For example, an illustrative assay measures Tat-regulated luciferase reporter gene expression to quantify the reduction of virus infection in TZM-bl cells (Montefiori, et al. Methods Mol. Biol. 485:395-405, 2009; Sarzotti-Kelsoe). The 50% inhibitory concentration (IC₅₀) is the concentration of antibody at which relative luminescence units are reduced by at least 50% as compared to infection in the absence of anti-HIV antibody, or in the presence of a negative control antibody after background is subtracted. In some embodiments, neutralizing activity can also be measured as a function of the area under the positive portion of the neutralization curve. Breadth and potency are two typical measures that may be employed to characterize an antibody's neutralizing activity. Breadth is the proportion of tested viruses with IC₅₀ scores that fall below an IC₅₀ cutoff value for neutralizing activity. Potency can be calculated using the geometric mean IC₅₀ (see, e.g., Hraber et al., J Virol. 88:12623-43, 2014; Rademeyer). In some embodiments, an anti-HIV variant antibody as described herein is at least 0.25, 0.3, 0.35, 0.4, 0.45, 0.5, 0.6, 0.7, 0.8, 0.9 times as potent, is equivalently potent, or is more potent, than the L1 Al, L1A4, or L2A1 antibody from which the variant is dervied when activity is compared in the same assay.

In some embodiments, binding activity of a variant anti-HIV antibody as described herein to HIV Env protein can be assessed. Binding can be determined using any immunoassay, where examples using recombinant gp120 may include ELISA, SPR, or similar assays. The gp120 protein can be from various HIV strains. In some embodiments, the HIV strain is BaL. In some embodiments, HIV binding is assessed by measuring binding to an HIV Env trimer in which the trimer is expressed on the surface of cells transfected with HIV Env protein, is on the surface of infected cells, or is added to an ELISA as purified trimeric protein with or without the stabilizing SOSIP modification.

In some embodiments, binding of a variant anti-HIV antibody to HIV Env protein is assessed in a competitive assay format with a reference antibody, i.e., L1A1, L1A4, or L2A1 from which the variant is derived; or a reference antibody having the variable regions of the reference antibody. In some embodiments, a variant anti-HIV antibody in accordance with the present disclosure may block binding of the reference antibody in a competition assay by about 50% or more.

Anti-HIV antibodies of the present disclosure may also be evaluated in various assays for their ability to mediate FcR-dependent activity. Such assays are routine in the art. In some embodiments, antibody-dependent cellular cytotoxicity (ADCC) is measured. In some embodiments, antibody-dependent cellular viral inhibition (ADCVI) is measured. For example, ADCC can be measured by quantifying the destruction of Env-coated or HIV-infected fluorescent cells driven by the addition of either PBMCs or specifc effector cell populations such as NK cells. In such an analysis, ADCC activity is reported as a reduction in percent of Env-coated or HIV-infected cells in the presence and absence of anti-HIV antibodies and effector cells. ADCVI is measured by quantifying the amount of virus produced by infected cells in the presence and absence of anti-HIV antibody and PBMCs. ADCVI is frequently reported as a reduction in p24 measured in the cellular supernatant. In some embodiments, an anti-HIV variant antibody of the present disclosure has enhanced ADCC and/or ADCVI activity compared to antibody L1A1, L1A4, or L2A1 from which the variant is derived when the antibodies are assayed in a human IgG1 isotype format.

### Generation of Antibodies

Anti-HIV antibodies as disclosed herein are commonly produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. Accordingly, in a further aspect of the invention, provided herein are isolated nucleic acids encoding a V_{H} and/or V_{L} region, or fragment thereof, of any of the anti-HIV antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the anti-HIV antibody (e.g., the light and/or heavy chains of the antibody). In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

In a further aspect, the invention provides a method of making an anti-HIV antibody as described herein. In some embodiments, the method includes culturing a host cell as described in the preceding paragraph under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing an anti-HIV antibody as described herein include both prokaryotic or eukaryotic cells. For example, anti-HIV antibodies may be produced in bacteria, in particular when glycosylation and Fc effector function are not needed. After expression, the antibody may be isolated from the bacterial cell paste in a soluble fraction and can be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing anti-HIV antibodies of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells may be used to express anti-HIV1 antibodies. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

A host cell transfected with an expression vector encoding an anti-HIV antibody of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, provided herein is a method of generating variants of an anti-HIV antibody of the present invention. Thus, for example, a construct encoding a variant L1A1, L1A4, or L2A1 Vx CDR3 as described in the sections above can be additionally modified and the V_{H} region encoded by the additionally modified construct can be tested for gp120 binding activity and/or neutralizing activity in the context of a V_{H} region comprising the native CDR1 and CDR2, or a variant CDR1 or CDR2, as described herein that is paired with a native or variant corresponding V_{L} region as described herein. Similarly, a construct encoding a variant L1A1, L1A4, or L2A1 V_{L} CDR3 as described in the sections above on can be additionally modified and the V_{L} region encoded by the additionally modified construct can be tested for gp120 binding activity and/or neutralizing activity in the context of a V_{L} region comprising the native CDR1 and CDR2, or a variant CDR1 or CDR2, as described herein that is paired with a native or variant corresponding V_{H} region as described herein. Such an analysis can also be performed with other CDRs or framework regions and an antibody having the desired activity can then be selected.

### Anti-HIV Antibody Conjugates

In a further aspect, an anti-HIV antibody of the present invention may be conjugated or linked to therapeutic and/or imaging/detectable moieties. For example, the anti-HIV antibody may be conjugated to a detectable marker, a toxin, or a therapeutic agent. Methods for conjugating or linking antibodies are well known in the art. The moiety may be linked to the antibody covalently or by non-covalent linkages.

In some embodiments, the antibody is conjugated to cytotoxic moiety or other moiety that inhibits cell proliferation. In some embodiments, the antibody is conjugated to a cytotoxic agent including, but not limited to, a ricin A chain, doxorubicin, daunorubicin, a maytansinoid, taxol, ethidium bromide, mitomycin, etoposide, tenoposide, vincristine, vinblastine, colchicine, dihydroxy anthracin dione, actinomycin, a diphtheria toxin, extotoxin A from Pseudomonas, Pseudomonas exotoxin (PE) A, PE40, abrin, abrin A chain, modeccin A chain, alpha sarcin, gelonin, mitogellin, restrictocin, cobran venom factor, a ribonuclease, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a roliferation inhibitor, a lytic agent, a DNA or RNA synthesis inhibitors, a membrane permeability modifier, a DNA metabolites, a dichloroethylsulfide derivative, a protein production inhibitor, a ribosome inhibitor, or an inducer of apoptosis.

In some embodiments, the antibody may be linked to radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

### Pharmaceutical Compositions

In a further aspect, provided herein are pharmaceutical compositions for administration of an anti-HIV antibody of the present invention to a mammalian subject, preferably a human or non-human primate subject, that is infected with HIV or is at risk of HIV infection, in an amount and according to a schedule sufficient to prevent HIV infection or reduce HIV viral load in the subject. Such compositions may comprise an anti-HIV-1 antibody or a polynucleotide encoding the antibody, and a pharmaceutically acceptable diluent or carrier. In some embodiments, the polynucleotide encoding the antibody may be contained in a plasmid vector for delivery, or a viral vector. In some embodiments, the pharmaceutical composition comprises a therapeutically effective amount of the antibody. As used herein, a “therapeutically effective dose” or a “therapeutically effective amount” refers to an amount sufficient to prevent, cure, or at least partially arrest HIV infection or symptoms of HIV infection and its complications. A therapeutically effective dose can be determined by monitoring a patient's response to therapy. Typical benchmarks indicative of a therapeutically effective dose include amelioration of symptoms of the disease in the patient, including, for example, reduction in viral load and increases in CD4+ lymphocyte numbers. Amounts effective for this use will depend upon the severity of the disease and the general state of the patient's health, including other factors such as age, weight, gender, administration route, etc. Single or multiple administrations of the antibody are dependent on the dosage and frequency as required and tolerated by the patient.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in Remington: The Science and Practice of Pharmacy 20th Ed. (Lippincott, Williams & Wilkins 2012). In particular embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., phosphate-buffered saline; sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The pharmaceutical composition can be formulated for any suitable route of administration, including for example, parenteral, intrapulmonary, intranasal, or local administration. Parenteral administration can include intramuscular, intravenous, intraarterial, intraperitoneal, oral or subcutaneous administration. In certain embodiments, the pharmaceutical composition is formulated for intravenous administration and has a concentration of antibody of 10-100 mg/ml, 10-50 mg/ml, 20 to 40 mg/ml, or about 30 mg/ml. In certain embodiments, the pharmaceutical composition is formulated for subcutaneous injection and has a concentration of antibody of 50-500 mg/ml, 50-250 mg/ml, or 100 to 150 mg/ml, and a viscosity less than 50 cP, less than 30 cP, less than 20 cP, or about 10 cP. In some embodiments, the pharmaceutical compositions are liquids or solids. In particular embodiments, the pharmaceutical compositions are formulated for parenteral, e.g., intravenous, subcutaneous, or oral administration.

The formulation of and delivery methods of pharmaceutical compositions will generally be adapted according to the site and the disease to be treated. Formulations include those in which the antibody is encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays.

In some embodiments, e.g., for parenteral administration, the antibodies or antigen-binding fragments thereof are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate may also be used.

The dose and dosage regimen depends upon a variety of factors readily determined by a physician, such as the nature of the infection, the characteristics of the subject, and the subject's history. In particular embodiments, the amount of antibody or antigen-binding fragment thereof administered or provided to the subject is in the range of about 0.1 mg/kg to about 50 mg/kg of the subject's body weight. Depending on the type and severity of the infection, in certain embodiments, about 0.1 mg/kg to about 50 mg/kg body weight (e.g., about 0.1-15 mg/kg/dose) of antibody or antigen-binding fragment thereof may be provided as an initial candidate dosage to the patient, whether, for example, by one or more separate administrations, or by continuous infusion. The progress of the therapy is readily monitored by conventional methods and assays and based on criteria known to the physician or other persons of skill in the art.

### Methods of Treating or Preventing HIV Infection

In a further aspect, provided herein are methods of treating and/or preventing an HIV infection or complication of an HIV infection, the method comprising administering to a subject, e.g., a human or non-human primate, in need thereof an effective amount of an anti-HIV antibody as described herein, or a polynucleotide encoding such an antibody. In some embodiments, the antibody is administered to an individual at risk of acquiring an HIV infection. In some embodiments, the antibody is administered to a patient who has acquired immune deficiency syndrome (AIDS). In some embodiments, the subject is a virologically suppressed HIV-infected mammal, such as a human or non-human primate, while in other embodiments, the subject is a treatment-naive HIV-infected mammal. In certain embodiments, a treatment-naive subject has a viral load between 10³ and 10⁵ copies/ml, and in certain embodiments, a virologically suppressed subject has a viral load <50 copies/ml. In some embodiments, the subject is a human. In certain embodiments, the subject has been diagnosed with an HIV, e.g., HIV-1 or HIV-2, infection or a related disease or disorder, e.g., AIDS, or is considered at risk for contracting an HIV, e.g., HIV-1 or HIV-2, infection and/or developing a related disease or disorder, e.g., AIDS. Subjects at risk for HIV infection include individuals who have come into contact with an infected person or who have been exposed to HIV in some other way. Administration of the antibody can occur prior to exposure such that infection or disease is prevented, or can be administered following infection to prevent, delay, and/or reduce manifestation of symptoms characteristic of HIV-related disease or disorders.

The present invention further provides methods for preventing or inhibiting an increase in HIV virus titer, virus replication, virus proliferation or an amount of an HIV viral RNA, HIV viral DNA, HIV proviral DNA, or HIV viral protein in a subject. In one embodiment, the method comprises providing to the subject in need thereof an amount of an antibody effective to prevent an increase in HIV viral load, virus replication or an amount of an HIV protein of one or more HIV strains or isolates in the subject. In certain embodiments, the method further comprises measuring an amount of HIV viral or RNA, DNA, or proviral DNA or protein at one or more time points, e.g., before and after the subject is administered the antibody or one or more polynucleotides.

An antibody of the present disclosure may be administered to a subject using any route of administration, e.g., systemic, parenterally, locally, in accordance with known methods. Such routes include, but are not limited to, intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. A subject may be administered an antibody of the present invention one or more times; and may be administered before, after, or concurrently with another therapeutic agent as further described below.

In certain embodiments, the antibody or antigen-binding fragment thereof of the present invention is provided to the subject in combination with one or more additional therapeutic agents used to treat HIV infection or a related disease or disorder. In certain embodiments, a method for treating or preventing an HIV infection in a mammal, e.g., a human, having or at risk of having the infection is provided, comprising administering to the human a therapeutically effective amount of an antibody as disclosed herein, or a pharmaceutically acceptable salt thereof, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of an antibody as disclosed herein, or a pharmaceutically acceptable salt thereof, in combination with a therapeutically effective amount of one or more (e.g., one, two, three, one or two, or one to three) additional therapeutic agents.

In some embodiments, two or more antibodies of the present disclosure may be administered to the subject. In some embodiments, the two or more antibodies may have different neutralization capabilities, i.e., they exhibit a different neutralization profiles for different HIV strain or combinations of strains, as compared to each other. In some embodiments, the antibody may be administered with another anti-HIV therapeutic antibody.

In some embodiments, an additional therapeutic agent may be an anti-HIV agent.

For example, in some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, HIV maturation inhibitors, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies (e.g., PD-1 modulators, PD-L1 modulators, toll like receptors modulators, IL-15 agonists), other HIV antibodies, bispecific antibodies and “antibody-like” therapeutic proteins (e.g., DARTs®, Duobodies®, Bites®, XmAbs®, TandAbs®, Fab derivatives) including those targeting HIV gp120 or gp41, combination drugs for HIV, HIV p17 matrix protein inhibitors, IL-13 antagonists, Peptidyl-prolyl cis-trans isomerase A modulators, Protein disulfide isomerase inhibitors, Complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, Integrin antagonists, Nucleoprotein inhibitors, Splicing factor modulators, COMM domain containing protein 1 modulators, HIV Ribonuclease H inhibitors, Retrocyclin modulators, CDK-9 inhibitors, Dendritic ICAM-3 grabbing nonintegrin 1 inhibitors, HIV GAG protein inhibitors, HIV POL protein inhibitors, Complement Factor H modulators, Ubiquitin ligase inhibitors, Deoxycytidine kinase inhibitors, Cyclin dependent kinase inhibitors Proprotein convertase PC9 stimulators, ATP dependent RNA helicase DDX3X inhibitors, reverse transcriptase priming complex inhibitors, HIV gene therapy, PI3K inhibitors, compounds such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US20140221378 (Japan Tobacco), WO 2010/130034 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences) and WO 2013/006792 (Pharma Resources), and other drugs for treating HIV, and combinations thereof. In some embodiments, the additional therapeutic is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In some embodiments, the additional therapeutic agent is a latency reversing agent (LRA), e.g., a TLR7 agonist. In other embodiments, the additional therapeutic agent is a latency reversing agent (LRA), e.g., a TLR8 agonist. Examples of TLR agonists include but are not limited to Vesatolimod. Additional examples include but are not limited to the compounds described in U.S. Pat. No. 8,367,670 and the compounds described in U.S. Patent Application Publication No. 2016-0289229. In one embodiment, the antibody of the present invention may be combined with TLR7 agonist such as Vesatolimod. In another embodiment, the antibody of the present invention may be combined with TLR8 agonist. In one embodiment, the additional therapeutic agent is a TLR modulator. TLR modulators may include modulators of TLR1, TLR2, TLR3, TLR4, TLR5, TLR6, TLR7, TLR8, TLR9, TLR 10, TLR11, TLR 12, and TLR13. Examples of TLR3 modulators include rintatolimod, poly-ICLC, RIBOXXON®, Apoxxim, RIBOXXIM®, IPH-33, MCT-465, MCT-475, and ND-1.1. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences). Examples of TLR8 modulators include motolimod, resiquimod, 3M-051, 3M-052, MCT-465, IMO-4200, VTX-763, VTX-1463, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Examples of TLR9 modulators include BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10.

In some embodiments, the additional therapeutic agents comprise one or more antiretroviral therapies (ARTs). In particular embodiments, the ART is a combination ART (cART) such as highly active ART (HAART). In particular embodiments, the ART comprises one or more of a nucleoside reverse transcriptase inhibitor (NRTI), a non-nucleoside reverse transcriptase inhibitor (NNRTI), a protease inhibitor (PI), an entry inhibitor, or an HIV integrase inhibitor. Examples of NRTIs include but are not limited to: Zidovudine (Retrovir, AZT); Didanosine (Videx, Videx EC, ddl); Stavudine (Zerit, d4T); Lamivudine (Epivir, 3TC); Tenofovir, a nucleotide analog (Viread, TDF); Combivir (combination of zidovudine and lamivudine); Trizivir (combination of zidovudine, lamivudine and abacavir); Emtricitabine (Emtriva, FTC); Truvada (combination of emtricitabine and tenofovir); and Epzicom (combination of abacavir and lamivudine). Examples of NNRTIs include but are not limited to: Nevirapine (Viramune, NVP); Delavirdine (Rescriptor, DLV); Efavirenz (Sustiva or Stocrin, EFV, also part of Atripla); Etravirine (Intelence, ETR); and Rilpivirine (Edurant, RPV, also part of Complera or Epivlera). Examples of Pis include but are not limited to: Saquinavir (Invirase, SQV); Indinavir (Crixivan, IDV); Ritonavir (Norvir, RTV); Nelfinavir (Viracept, NFV); Amprenavir (Agenerase, APV); Lopinavir/ritonavir (Kaletra or Aluvia, LPV/RTV); Atazanavir (Reyataz, ATZ); Fosamprenavir (Lexiva, Telzir, FPV); Tipranavir (Aptivus, TPV); and Darunavir (Prezista, DRV). Examples of entry inhibitors include but are not limited to: Enfuvirtide (Fuzeon, ENF, T-20) and Maraviroc (Selzentry or Celsentri, MVC). Examples of HIV integras inhibitors include but are not limited to: Raltegravir (Isentress, RAL); Elvitegravir (EVG, part of the combination Stribild) and Dolutegravir (Tivicay, DTG).

In some embodiments, an anti-HIV antibody of the present invention is administered with a latency reversing agents (e.g., histone deacetylase inhibitors, proteasome inhibitors, protein kinase C (PKC) activators, bromo and external bromodomain inhibitors, acetaldehyde dehydrogenase inhibitors, and activators of nuclear factor kappa-light chain-enhancer of activated B cells (NF-κB) and the AKT pathway. In some embodiments, the latency reversing agent is a TLR7 agonist. In other embodiments, the latency reversing agent is a TLR8 agonist. Examples of TLR agonists include but are not limited to Vesatolimod. Additional examples include but are not limited to the compounds described in U.S. Pat. No. 8,367,670 and the compounds described in U.S. Patent Application Publication No. 2016-0289229. In one embodiment, the antibody of the present invention may be combined with TLR7 agonist such as Vesatolimod. In another embodiment, the antibody of the present invention may be combined with a TLR8 agonist. In one embodiment, the additional therapeutic agent is a TLR modulator. TLR modulators may include modulators of TLR1, TLR2, TLR3, TLR4, TLR5, TLR6, TLR7, TLR8, TLR9, TLR 10, TLR11, TLR 12, and TLR13. Examples of TLR3 modulators include rintatolimod, poly-ICLC, RIBOXXON®, Apoxxim, RIBOXXIM®, IPH-33, MCT-465, MCT-475, and ND-1.1. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences). Examples of TLR8 modulators include motolimod, resiquimod, 3M-051, 3M-052, MCT-465, IMO-4200, VTX-763, VTX-1463, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Examples of TLR9 modulators include BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10. In some embodiments, the latency reversing agent is a PKC agonist such as bryostatin-1, prostratin, ingenol-3-angelate, ingenol mimic, or DAG mimic. In certain embodiments, the latency reversing agent is an activator of NF-κB such as disulfiram. In certain embodiments, the latency reversing agent is a histone deacetylase inhibitor selected from the group consisting of vorinostat, panobinostat, and romidepsin. In other embodiments, the histone deacetylase inhibitor is selected from 4-phenylbutyrohydroxamic acid, Acetyldinaline, APHA, Apicidin, AR-42, Belinostat, CUDC-101, CUDC-907, Dacinostat, Depudecin, Droxinostat, Entinostat, Givinostat, HC-Toxin, ITF-2357, JNJ-26481585, KD 5170, LAQ-824, LMK 235, M344, MC1568, MGCD-0103, Mocetinostat, NCH 51, Niltubacin, NSC3852, Oxamflatin, Panobinostat, PCI-24781, PCI-34051, Pracinostat, Pyroxamide, Resminostat, RG2833, RGFP966, Rocilinostat, Romidepsin, SBHA, Scriptaid, Suberohydroxamic acid, Tacedinaline, TC-H 106, TCS HDAC6 20b, Tacedinaline, TMP269, Trichostatin A, Tubacin, Tubastatin A, Valproic acid, or Vorinostat. In certain embodiments, the latency reversing agent is a bromodomain inhibitor such as JQ1. In other embodiments, the inhibitor is selected from CPI 203, 1-BET151, 1-BET762, JQ1, MS417, MS436, OTX-015, PFi-1, or RVX-208. In certain embodiments, a combination of latency reversing agents is administered with an anti-HIV antibody of the present invention. The antibody may be administered simultaneously or sequentially, either before or after, with one or more latency reversing agents. In some embodiments, a subject may additionally undergo treatment with another therapeutic agent for HIV infection.

In some embodiments, an antibody of the present disclosure is formulated as a tablet, which may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In some embodiments, such tablets are suitable for once daily dosing.

In some embodiments, an antibody of the present disclosure is administered with an additional therapeutic agent selected from one or more of: (1) Combination drugs selected from the group consisting of ATRIPLA® (efavirenz+tenofovir disoproxil fumarate+emtricitabine), COMPLERA® (EVIPLERA®, rilpivirine+tenofovir disoproxil fumarate+emtricitabine), STRIBILD® (elvitegravir+cobicistat+tenofovir disoproxil fumarate+emtricitabine), dolutegravir+abacavir sulfate+lamivudine, TRIUMEQ® (dolutegravir+abacavir+lamivudine), lamivudine+nevirapine+zidovudine, dolutegravir+rilpivirine, dolutegravir+rilpivirine hydrochloride, atazanavir sulfate+cobicistat, atazanavir+cobicistat, darunavir+cobicistat, efavirenz+lamivudine+tenofovir disoproxil fumarate, tenofovir alafenamide hemifumarate+emtricitabine+cobicistat+elvitegravir, tenofovir alafenamide hemifumarate+emtricitabine, tenofovir alafenamide+emtricitabine, tenofovir alafenamide hemifumarate+emtricitabine+rilpivirine, tenofovir alafenamide+emtricitabine+rilpivirine, Vacc-4x+romidepsin, darunavir+tenofovir alafenamide hemifumarate+emtricitabine+cobicistat, APH-0812, raltegravir+lamivudine, KALETRA® (ALUVIA®, lopinavir+ritonavir), atazanavir sulfate+ritonavir, COMBIVIR® (zidovudine+lamivudine, AZT+3TC), EPZICOM® (Livexa®, abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), doravirine+lamivudine+tenofovir disoproxil fumarate, doravirine+lamivudine+tenofovir disoproxil, tenofovir+lamivudine and lamivudine+tenofovir disoproxil fumarate; (2) HIV protease inhibitors selected from the group consisting of amprenavir, atazanavir, fos amprenavir, fosamprenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100) and TMC-310911; (3) HIV non-nucleoside or non-nucleotide inhibitors of reverse transcriptase selected from the group consisting of delavirdine, delavirdine mesylate, nevirapine, etravirine, dapivirine, doravirine, rilpivirine, efavirenz, KM-023, VM-1500, lentinan and AIC-292; (4) HIV nucleoside or nucleotide inhibitors of reverse transcriptase selected from the group consisting of VIDEX® and VIDEX® EC (didanosine, ddl), zidovudine, emtricitabine, didanosine, stavudine, zalcitabine, lamivudine, censavudine, abacavir, abacavir sulfate, elvucitabine, alovudine, phosphazid, fozivudine tidoxil, apricitabine, KP-1461, fosalvudine tidoxil, tenofovir, tenofovir disoproxil, tenofovir disoproxil fumarate, tenofovir disoproxil hemifumarate, tenofovir alafenamide, tenofovir alafenamide hemifumarate, tenofovir alafenamide fumarate, adefovir, adefovir dipivoxil, and festinavir; (5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, elvitegravir, dolutegravir and cabotegravir; (6) HIV non-catalytic site, or allosteric, integrase inhibitors (NCINI) selected from the group consisting of CX-05168, CX-05045 and CX-14442; (7) HIV gp41 inhibitors selected from the group consisting of enfuvirtide, sifuvirtide and albuvirtide; (8) HIV entry inhibitors selected from the group consisting of cenicriviroc; (9) HIV gp120 inhibitors selected from the group consisting of Radha-108 (Receptol) and BMS-663068; (10) CCR5 inhibitors selected from the group consisting of aplaviroc, vicriviroc, maraviroc, cenicriviroc, PRO-140, Adaptavir (RAP-101), nifeviroc (TD-0232), TD-0680, and vMIP (Haimipu); (11) CD4 attachment inhibitors, e.g., Fostemsavir (BMS-663068); (12) inhibitors of post-binding events required for entry selected from the group consisting of ibalizumab and CXCR4 inhibitors such as plerixafor, ALT-1188, vMIP and Haimipu; (13) Pharmacokinetic enhancers selected from the group consisting of cobicistat and ritonavir; (14) Immune-based therapies selected from the group consisting of derma Vir, interleukin-7, plaquenil (hydroxychloroquine), proleukin (aldesleukin, IL-2), interferon alfa, interferon alfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-12, polymer polyethyleneimine (PEI), Gepon, VGV-1, MOR-22, BMS-936559, toll-like receptors modulators (TLR1, TLR2, TLR3, TLR4, TLR5, TLR6, TLR7, TLR8, TLR9, TLR10, TLR11, TLR1 2 and TLR13), rintatolimod and IR-103; (15) HIV vaccines selected from the group consisting of peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX), ALVAC HIV (vCP1521)/AIDSVAX B/E (gp120) (RV144), monomeric gp120 HIV-1 subtype C vaccine (Novartis), Remune, ITV-1, Contre Vir, Ad5-ENVA-48, DCVax-001 (CDX-2401), PEP-6409, Vacc-4x, Vacc-05, VAC-3S, multiclade DNA recombinant adenovirus-5 (rAd5), Pennvax-G, VRC-HIV MAB060-00-AB, AVX-101, AVX-201, HIV-LAMP-vax, Ad35, Ad35-GRIN, NAcGM3/VSSP ISA-51, poly-ICLC adjuvanted vaccines, Tatlmmune, GTU-multiHIV (FIT-06), AGS-004, gp140[delta]V2.TV1+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT 123, rAAV1-PG9DP, GOVX-B 11, GOVX-B21, ThV-01, TUTI-16, VGX-3300, TVI-HIV-1, Ad-4 (Ad4-env Clade C+Ad4-mGag), EN41-UGR7C, EN41-FPA2, PreVaxTat, TL-01, SAV-001, AE-H, MYM-V101, CombiHlVvac, ADVAX, MYM-V201, MVA-CMDR, MVATG-17401, ETV-01, CDX-1401, rcAd26.MOS 1.HIV-Env and DNA-Ad5 gag/pol/nef/nev (HVTN505); (16) HIV antibodies, bispecific antibodies and “antibody-like” therapeutic proteins (such as DARTs®, Duobodies®, Bites®, XmAbs®, TandAbs®, Fab derivatives) including BMS-936559, TMB-360 and those targeting HIV gp120 or gp41 selected from the group consisting of bavituximab, UB-421, C2F5, C2G12, C4E10, C2F5+C2G12+C4E10, 3-BNC-117, PGT145, PGT121, MDX010 (ipilimumab), VRCO1, A32, 7B2, 10E8, VRC-07-523 and VRC07; (17) latency reversing agents selected from the group consisting of Histone deacetylase inhibitors such as Romidepsin, vorinostat, panobinostat; Proteasome inhibitors such as Velcade; protein kinase C (PKC) activators such as Indolactam, Prostratin, Ingenol B and DAG-lactones, Ionomycin, GSK-343, PMA, SAHA, BRD4 inhibitors, IL-15, JQ1, disulfram, and amphotericin B; (18) HIV nucleocapsid p7 (NCp7) inhibitors selected from the group consisting of azodicarbonamide; (19) HIV maturation inhibitors selected from the group consisting of BMS-955176 and GSK-2838232; (20) PI3K inhibitors selected from the group consisting of idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401; (21) the compounds disclosed in WO 2004/096286 (Gilead Sciences), WO 2006/110157 (Gilead Sciences), WO 2006/015261 (Gilead Sciences), WO 2013/006738 (Gilead Sciences), US 2013/0165489 (University of Pennsylvania), US20140221380 (Japan Tobacco), US20140221378 (Japan Tobacco), WO 2013/006792 (Pharma Resources), WO 2009/062285 (Boehringer Ingelheim), WO 2010/130034 (Boehringer Ingelheim), WO 2013/091096A1 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences); and (22) other drugs for treating HIV selected from the group consisting of BanLec, MK-8507, AG-1105, TR-452, MK-8591, REP 9, CYT-107, alisporivir, NOV-205, IND-02, metenkefalin, PGN-007, Acemannan, Gamimune, Prolastin, 1,5-dicaffeoylquinic acid, BIT-225, RPI-MN, VSSP, Hlviral, IMO-3100, SB-728-T, RPI-MN, VIR-576, HGTV-43, MK-1376, rHIV7-shl-TAR-CCRSRZ, MazF gene therapy, BlockAide, ABX-464, SCY-635, naltrexone, AAV-eCD4-Ig gene therapy, TEV-90110, TEV-90112, deferiprone, and PA-1050040 (PA-040).

In certain embodiments, when an antibody of the present disclosure as described herein is combined with one or more additional therapeutic agents as described above, the components of the composition are administered as a simultaneous or sequential regimen. When administered sequentially, the combination may be administered in two or more administrations.

In some embodiments, an antibody as disclosed herein is combined with one or more additional therapeutic agents in a unitary dosage form for simultaneous administration to a patient, for example as a solid dosage form for oral administration.

“Co-administration” of an antibody as disclosed herein with one or more additional therapeutic agents generally refers to simultaneous or sequential administration of an antibody or fragment thereof disclosed herein and one or more additional therapeutic agents, such that therapeutically effective amounts of the antibody or fragment thereof disclosed herein and one or more additional therapeutic agents are both present in the body of the patient.

Co-administration includes administration of unit dosages of the antibody disclosed herein before or after administration of unit dosages of one or more additional therapeutic agents, for example, administration of the antibody within seconds, minutes, or hours of the administration of one or more additional therapeutic agents. For example, in some embodiments, a unit dose of an antibody disclosed herein is administered first, followed within seconds or minutes by administration of a unit dose of one or more additional therapeutic agents. Alternatively, in other embodiments, a unit dose of one or more additional therapeutic agents is administered first, followed by administration of a unit dose of an antibody within seconds or minutes. In some embodiments, a unit dose of an antibody disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of the antibody.

The combined administration may be co-administration, using separate pharmaceutical compositions or a single pharmaceutical composition, or consecutive administration in either order, wherein there is optionally a time period while both (or all) therapeutic agents simultaneously exert their biological activities. Such combined therapy may result in a synergistic therapeutic effect. In certain embodiments, it is desirable to combine administration of an antibody of the invention with another antibody directed against another antigen associated with the HIV infectious agent.

As described herein, the antibody may also be administered by gene therapy by administration of a nucleic acid comprising one or more polynucleotides encoding the antibody. In certain embodiments, the polynucleotide encodes an scFv. In particular embodiments, the polynucleotide comprises DNA, cDNA or RNA. In certain embodiments, the polynucleotide is present in a vector, e.g., a viral vector

The following examples are offered for illustrative purposes, and are not intended to limit the invention. Those of skill in the art will readily recognize a variety of non-critical parameters that can be changed or modified to yield essentially the same results.

### EXAMPLES

### Example 1. Generation of Anti-HIV Antibody Variants

Patient-derived broadly neutralizing antibodies against HIV were identified from a donor, NVS49, which included the lineages L1 and L2. L1 included antibodies L1A1, L1A2, and L1A4, among other antibodies. L2 included antibody L2A1, among other antibodies. This example describes antibody variants of antibodies L1A1, L1A4, and L2A1.

Variants were designed to remove liabilities that can cause undesirable antibody properties, resulting in delays in development, increased development costs, failure in development, or increased product costs. Desired antibody properties comprise: (1) suitable for a standard platform (expression, purification, formulation); (2) high yield; (3) low heterogeneity (glycosylation, chemical modification, etc.); (4) consistent manufacturability (batch-to-batch, and small-to-large scale); (5) high stability (years in liquid formulation), e.g., minimal chemical degradation, fragmentation, and aggregation; and (6) long PK (in vivo half life), e.g., no off-target binding, no impairment of FcRn recycling, and stable. Illustrative motifs considered during design of variants included the following. The “Risk” category is assigned in this table based on the likelihood of having the impact shown in Column 2.

TABLE 3
Description of potential development liabilities

| Property | Potential Impact | Seq. motif | Risk |
|---|---|---|---|
| Free cysteine | Yield, | Odd # C | High |
| | heterogeneity, | | |
| | stability, activity | | |
| N-linked | Yield, | N(~P)(S, T) | High |
| glycosylation | heterogeneity, | | |
| | activity | | |
| Abnormal net | Platform fit, PK | [Sharma 2014] | High |
| charge | | | |
| Patches of | Stability, PK | [Sharma 2014] | High |
| hydrophobicity | | | |
| Patches of | Stability, PK | N/A (based | Medium |
| same charge | | on structure) | |
| Proteolysis | Stability, PK | (K, R)(K, R) | Medium |
| Proteolysis | Stability, PK | DP | Medium |
| Asparagine | Heterogeneity, | NG; | Medium; |
| deamidation | stability, activity | N(A, N, S, T)* | Low |
| Aspartate | Heterogeneity, | DG; | Medium; |
| isomerization | stability, activity | D(A, D, S, T)* | Low |
| Lysine glycation | Heterogeneity, | K | Low |
| | stability, activity | | |
| Methionine | Heterogeneity, | M | Low |
| oxidation | stability, activity | | |
| Tryptophan | Heterogeneity, | W | Low |
| oxidation | stability, activity | | |

*N and D are also potentially low risk for other successor residues, e.g., D, N, H, or P

Another goal in generating variants is to reduce the risk of clinical immunogenicity and the generation of anti-drug antibodies (ADAs) against the therapeutic antibody. To minimize risk of immunogenicity, the lead sequence can be engineered to be as similar to the intended patient population's native (“self”) sequences as possible.

One approach employed to design variants that are as much like self as possible was to identify the closest germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all of the variable heavy (VH) and variable light (VL) regions except for part of H-CDR3. Germline gene IGHD codes for part of the H-CDR3 region but typically exhibits too much variation in how it is recombined with IGHV and IGHJ (eg, forward or reverse orientation, any of three translation frames, and 5′ and 3′ modifications and non-templated additions) to present a “self” sequence template from a population perspective.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for the prediction of HLA binding, and use in vitro assessment to further identify potential epitopes.

Development liabilities can be removed or reduced by one or more mutations. In the absence of any additional information (eg, sibling sequences or protein structure), mutations should aim to at least preserve antibody structure and function while removing or reducing the liability. Mutations to chemically similar residues are a preferred approach, eg, maintaining size, shape, charge, and/or polarity. Illustrative mutations are described in Table 4.

TABLE 4
Potential development liabilities and
illustrative mutations to reduce risk

| | Seq. motif | | Illustrative |
|---|---|---|---|
| Property | (CDR) | Risk | mutations |
| Free cysteine | Odd # C | High | C→(A, S) |
| N-linked | N(~P)(S, T) | High | N→(Q, D, S, A); |
| glycosylation | | | (S, T)→(A, N) |
| Proteolytic | (K, R)(K, R) | Medium | K, R→(Q, S, A) |
| cleavage | | | |
| Proteolytic | DP | Medium | D→(E, S, A) |
| cleavage | | | |
| Asparagine | NG; | Medium; | N→(Q, S, A); G→(A, S) |
| deamidation | N(A, N, S, T)* | Low | |
| Aspartate | DG; | Medium; | D→(E, S, A); G→(A, S) |
| isomerization | D(A, D, S, T)* | Low | |
| Lysine glycation | K | Low | K→(R, Q, S, A) |
| Methionine | M | Low | M→(Q, L, S, A) |
| oxidation | | | |
| Tryptophan | W | Low | W→(Y, F) |
| oxidation | | | |

Variants of L1A1 and L1A4 were also designed taking into consideration the sequences of siblings, including L1A2.

### Assessment of NVS49-L1 and NVS49-L2 Lineages

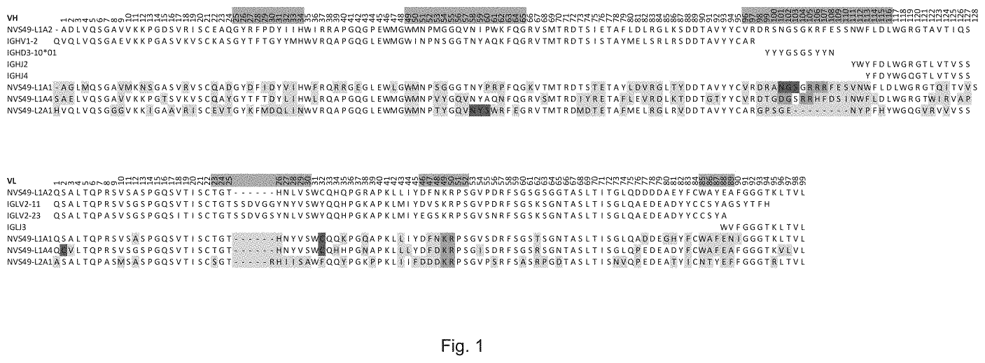

The VH and VL regions sequences of L1A1, L1A4, and L2A1 were aligned to each other, using antibody L1A2 as reference (FIG. 1). The closest germline sequences (HV1-2, HD3-10*01, HJ2 & HJ4, LV2-11 & LV2-23, and LJ3) were also aligned. CDRs, germline deviations, and potential liabilities were all identified. Non-canonical cysteines and N-glycosylation sites were identified across the full VH and VL, whereas the other potential liability motifs were identified only within the CDRs.

Potential PK risk was also estimated based Sharma et al., Proc. Natl. Acad. Sci. USA 111:18601-18606, 2014/High hydrophobicity index (HI) was found to correlate with faster clearance, where HI<5 is preferred to reduce risk, and HI<4 is most preferred to reduce risk. However, some antibodies with HI >4, or HI >5, will not exhibit fast clearance, and be false positives. Secondly, too high or too low Fv charge as calculated at pH 5.5 was found to correlate with faster clearance, where charge between (−2, +8) is preferred to reduce risk, and charge between (0, +6.2) is most preferred to reduce risk.

TABLE 5
Summary of potential liabilities L1A1, L1A4, and L2A3

| | L1A1 | L1A4 | L2A1 |
|---|---|---|---|
| Odd # C | 1 | 2 | 0 |
| N(~P)(S, T) | 1 | 0 | 1 |
| Fv charge 5.5 | 6.95 | 5.56 | 3.30 |
| HI | 3.50 | 6.31 | 2.84 |
| (K, R)(K, R) | 3 | 2 | 1 |
| DP | 0 | 0 | 0 |
| NG | 1 | 0 | 0 |
| N(A, N, S, T) | 0 | 0 | 1 |
| DG | 0 | 1 | 0 |
| D(A, D, S, T) | 0 | 2 | 2 |
| K | 1 | 1 | 2 |
| M | 1 | 1 | 2 |
| W | 3 | 3 | 2 |
| FW non-germ | 39 | 40 | 40 |
| VH | 28 | 27 | 21 |
| VL | 11 | 13 | 19 |

### Design of Variants of L1A1, L1A4, and L2A1

Variants of antibodies L1A1, L1A4, or L2A1, were designed to reduce development liabilities, reduce immunogenicity risk, and enhance function. Designed variants are shown in Table, 6, 7, and 8, respectively. Histidine is approximated as +0.5 charge because its side chain pKa is near physiological pH.

TABLE 6
L1A1 variable positions

| | | | | | | Mutation in |
|---|---|---|---|---|---|---|
| | | | | Risk of | Change | an L1A2 |
| | | | | leaving | in | OR L1A4 |
| Parent | Mutation | Purpose(s) | Location | as native | charge | sibling? |
| L1A1 | H0XQ | germlining | H-FW1 | medium | 0.0 | no |
| L1A1 | H1AV | germlining | H-FW1 | medium | 0.0 | no |
| L1A1 | H2GQ | germlining | H-FW1 | medium | 0.0 | no |
| L1A1 | H4MV | germlining | H-FW1 | medium | 0.0 | yes |
| L1A1 | H9VE | germlining | H-FW1 | medium | −1.0 | no |
| L1A1 | H10MV | germlining | H-FW1 | medium | 0.0 | yes |
| L1A1 | H12NK | germlining | H-FW1 | medium | +1.0 | yes |
| L1A1 | H13SP | germlining | H-FW1 | medium | 0.0 | yes |
| L1A1 | H18RK | germlining | H-FW1 | medium | 0.0 | yes |
| L1A1 | H22QK | germlining | H-FW1 | medium | +1.0 | no |
| L1A1 | H24DS | germlining | H-FW1 | medium | +1.0 | no |
| L1A1 | H36FV | germlining | H-FW2 | medium | 0.0 | no |
| L1A1 | H39RA | germlining | H-FW2 | medium | −1.0 | yes |
| L1A1 | H40RP | germlining | H-FW2 | medium | −1.0 | yes |
| L1A1 | H42EQ | germlining | H-FW2 | medium | +1.0 | yes |
| L1A1 | H47LM | germlining | H-FW2 | medium | 0.0 | yes |
| L1A1 | H49WY | liab: Trp | H-CDR2 | low | 0.0 | no |
| | | oxidation | | | | |
| L1A1 | H49WF | liab: Trp | H-CDR2 | low | 0.0 | no |
| | | oxidation | | | | |
| L1A1 | H50MI | liab: Met | H-CDR2 | low | 0.0 | no |
| | | oxidation | | | | |
| L1A1 | H50MQ | liab: Met | H-CDR2 | low | 0.0 | no |
| | | oxidation | | | | |
| L1A1 | H50ML | liab: Met | H-CDR2 | low | 0.0 | no |
| | | oxidation | | | | |
| L1A1 | H50MS | liab: Met | H-CDR2 | low | 0.0 | no |
| | | oxidation | | | | |
| L1A1 | H50MA | liab: Met | H-CDR2 | low | 0.0 | no |
| | | oxidation | | | | |
| L1A1 | H66KR | germlining | H-FW3 | medium | 0.0 | yes |
| L1A1 | H75TI | germlining | H-FW3 | medium | 0.0 | yes |
| L1A1 | H76ES | germlining | H-FW3 | medium | +1.0 | no |
| L1A1 | H80LM | germlining | H-FW3 | medium | 0.0 | no |
| L1A1 | H81DE | germlining | H-FW3 | medium | 0.0 | yes |
| L1A1 | H82VL | germlining | H-FW3 | medium | 0.0 | yes |
| L1A1 | H83RS | germlining | H-FW3 | medium | −1.0 | no |
| L1A1 | H84GR | germlining | H-FW3 | medium | +1.0 | no |
| L1A1 | H86TR | germlining | H-FW3 | medium | +1.0 | no |
| L1A1 | H87YS | germlining | H-FW3 | medium | 0.0 | yes |
| L1A1 | H101ND | liab: Nglyco | H-CDR3 | high | −1.0 | yes |
| L1A1 | H101NS | liab: Nglyco, | H-CDR3 | high | 0.0 | no |
| | | NG deamidation | | | | |
| L1A1 | H101NA | liab: Nglyco, | H-CDR3 | high | 0.0 | no |
| | | NG deamidation | | | | |
| L1A1 | H101NQ | liab: Nglyco, | H-CDR3 | high | 0.0 | no |
| | | NG deamidation | | | | |
| L1A1 | H103SW | liab: Nglyco | H-CDR3 | high | 0.0 | no |
| L1A1 | H103SA | liab: Nglyco | H-CDR3 | high | 0.0 | no |
| L1A1 | H103SN | liab: Nglyco | H-CDR3 | high | 0.0 | no |
| L1A1 | H105RQ | liab: | H-CDR3 | medium | −1.0 | no |
| | | proteolysis | | | | |
| L1A1 | H105RS | liab: | H-CDR3 | medium | −1.0 | no |
| | | proteolysis | | | | |
| L1A1 | H105RA | liab: | H-CDR3 | medium | −1.0 | no |
| | | proteolysis | | | | |
| L1A1 | H106RQ | liab: | H-CDR3 | medium | −1.0 | no |
| | | proteolysis | | | | |
| L1A1 | H106RS | liab: | H-CDR3 | medium | −1.0 | no |
| | | proteolysis | | | | |
| L1A1 | H106RA | liab: | H-CDR3 | medium | −1.0 | no |
| | | proteolysis | | | | |
| L1A1 | H107RQ | liab: | H-CDR3 | medium | −1.0 | no |
| | | proteolysis | | | | |
| L1A1 | H107RS | liab: | H-CDR3 | medium | −1.0 | no |
| | | proteolysis | | | | |
| L1A1 | H107RA | liab: | H-CDR3 | medium | −1.0 | no |
| | | proteolysis | | | | |
| L1A1 | H113WY | liab: Trp | H-CDR3 | low | 0.0 | no |
| | | oxidation | | | | |
| L1A1 | H113WF | liab: Trp | H-CDR3 | low | 0.0 | no |
| | | oxidation | | | | |
| L1A1 | H123QL | germlining | H-FW4 | medium | 0.0 | no |
| L1A1 | H124IV | germlining | H-FW4 | medium | 0.0 | no |
| L1A1 | H127VS | germlining | H-FW4 | medium | 0.0 | yes |
| L1A1 | L12AG | germlining | L-FW1 | medium | 0.0 | yes |
| L1A1 | L32CY | liab: cys & | L-FW2 | high | 0.0 | no |
| | | germlining | | | | |
| L1A1 | L32CA | liab: cys | L-FW2 | high | 0.0 | no |
| L1A1 | L32CV | liab: cys | L-FW2 | high | 0.0 | no |
| L1A1 | L32CL | liab: cys | L-FW2 | high | 0.0 | no |
| L1A1 | L32CI | liab: cys | L-FW2 | high | 0.0 | no |
| L1A1 | L35KH | germlining | L-FW2 | medium | −0.5 | yes |
| L1A1 | L38QK | germlining | L-FW2 | medium | +1.0 | no |
| L1A1 | L43LM | germlining | L-FW2 | medium | 0.0 | no |
| L1A1 | L49KQ | liab: | L-CDR2 | medium | −1.0 | no |
| | | proteolysis | | | | |
| L1A1 | L49KS | liab: | L-CDR2 | medium | −1.0 | no |
| | | proteolysis | | | | |
| L1A1 | L49KA | liab: | L-CDR2 | medium | −1.0 | no |
| | | proteolysis | | | | |
| L1A1 | L50RQ | liab: | L-CDR2 | medium | −1.0 | no |
| | | proteolysis | | | | |
| L1A1 | L50RS | liab: | L-CDR2 | medium | −1.0 | no |
| | | proteolysis | | | | |
| L1A1 | L50RA | liab: | L-CDR2 | medium | −1.0 | no |
| | | proteolysis | | | | |
| L1A1 | L62TK | germlining | L-FW3 | medium | +1.0 | no |
| L1A1 | L77DE | germlining | L-FW3 | medium | 0.0 | yes |
| L1A1 | L80GA | germlining | L-FW3 | medium | 0.0 | yes |
| L1A1 | L81HD | germlining | L-FW3 | medium | −1.5 | yes |
| L1A1 | L83FY | germlining | L-FW3 | medium | 0.0 | no |
| L1A1 | L85WY | liab: Trp | L-CDR3 | low | 0.0 | no |
| | | oxidation | | | | |
| L1A1 | L85WF | liab: Trp | L-CDR3 | low | 0.0 | no |
| | | oxidation | | | | |
| L1A1 | L90IF | germlining | L-FW4 | medium | 0.0 | yes |

TABLE 7
L1A4 variable positions

| | | | | | | Mutation in |
|---|---|---|---|---|---|---|
| | | | | Risk of | Change | an L1A1 |
| | | | | leaving | in | or L1A2 |
| Parent | Mutation | Purpose(s) | Location | as native | charge | sibling? |
| L1A4 | H0SQ | germlining | H-FW1 | medium | 0.0 | no |
| L1A4 | H1AV | germlining | H-FW1 | medium | 0.0 | no |
| L1A4 | H2EQ | germlining | H-FW1 | medium | +1.0 | no |
| L1A4 | H9VE | germlining | H-FW1 | medium | −1.0 | no |
| L1A4 | H15TA | germlining | H-FW1 | medium | 0.0 | yes |
| L1A4 | H22QK | germlining | H-FW1 | medium | +1.0 | no |
| L1A4 | H24YS | germlining | H-FW1 | medium | 0.0 | no |
| L1A4 | H36LV | germlining | H-FW2 | medium | 0.0 | no |
| L1A4 | H49WY | liab: Trp | H-CDR2 | low | 0.0 | no |
| | | oxidation | | | | |
| L1A4 | H49WF | liab: Trp | H-CDR2 | low | 0.0 | no |
| | | oxidation | | | | |
| L1A4 | H50MI | liab: Met | H-CDR2 | low | 0.0 | no |
| | | oxidation | | | | |
| L1A4 | H50MQ | liab: Met | H-CDR2 | low | 0.0 | no |
| | | oxidation | | | | |
| L1A4 | H50ML | liab: Met | H-CDR2 | low | 0.0 | no |
| | | oxidation | | | | |
| L1A4 | H50MS | liab: Met | H-CDR2 | low | 0.0 | no |
| | | oxidation | | | | |
| L1A4 | H50MA | liab: Met | H-CDR2 | low | 0.0 | no |
| | | oxidation | | | | |
| L1A4 | H68ST | germlining | H-FW3 | medium | 0.0 | yes |
| L1A4 | H73IT | germlining | H-FW3 | medium | 0.0 | yes |
| L1A4 | H74YS | germlining | H-FW3 | medium | 0.0 | yes |
| L1A4 | H75RI | germlining | H-FW3 | medium | −1.0 | yes |
| L1A4 | H76ES | germlining | H-FW3 | medium | +1.0 | no |
| L1A4 | H79FY | germlining | H-FW3 | medium | 0.0 | yes |
| L1A4 | H80LM | germlining | H-FW3 | medium | 0.0 | no |
| L1A4 | H82VL | germlining | H-FW3 | medium | 0.0 | yes |
| L1A4 | H83RS | germlining | H-FW3 | medium | −1.0 | no |
| L1A4 | H84DR | germlining | H-FW3 | medium | +2.0 | no |
| L1A4 | H86KR | germlining | H-FW3 | medium | 0.0 | no |
| L1A4 | H87TS | germlining | H-FW3 | medium | 0.0 | yes |
| L1A4 | H91GA | germlining | H-FW3 | medium | 0.0 | yes |
| L1A4 | H92TV | germlining | H-FW3 | medium | 0.0 | yes |
| L1A4 | H98DE | liab: DT | H-CDR3 | low | 0.0 | no |
| | | isomerization | | | | |
| L1A4 | H98DS | liab: DT | H-CDR3 | low | +1.0 | no |
| | | isomerization | | | | |
| L1A4 | H98DA | liab: DT | H-CDR3 | low | +1.0 | no |
| | | isomerization | | | | |
| L1A4 | H99TR | liab: DT | H-CDR3 | low | +1.0 | yes |
| | | isomerization | | | | |
| L1A4 | H99TK | liab: DT | H-CDR3 | low | +1.0 | no |
| | | isomerization | | | | |
| L1A4 | H101DE | liab: DG | H-CDR3 | medium | 0.0 | no |
| | | isomerization | | | | |
| L1A4 | H101DS | liab: DG | H-CDR3 | medium | +1.0 | no |
| | | isomerization | | | | |
| L1A4 | H101DA | liab: DG | H-CDR3 | medium | +1.0 | no |
| | | isomerization | | | | |
| L1A4 | H102GA | liab: DG | H-CDR3 | medium | 0.0 | no |
| | | isomerization | | | | |
| L1A4 | H102GS | liab: DG | H-CDR3 | medium | 0.0 | no |
| | | isomerization | | | | |
| L1A4 | H104RQ | liab: | H-CDR3 | medium | −1.0 | no |
| | | proteolysis | | | | |
| L1A4 | H104RS | liab: | H-CDR3 | medium | −1.0 | no |
| | | proteolysis | | | | |
| L1A4 | H104RA | liab: | H-CDR3 | medium | −1.0 | no |
| | | proteolysis | | | | |
| L1A4 | H104RG | liab: | H-CDR3 | medium | −1.0 | yes |
| | | proteolysis | | | | |
| L1A4 | H105RQ | liab: | H-CDR3 | medium | −1.0 | no |
| | | proteolysis | | | | |
| L1A4 | H105RS | liab: | H-CDR3 | medium | −1.0 | no |
| | | proteolysis | | | | |
| L1A4 | H105RA | liab: | H-CDR3 | medium | −1.0 | no |
| | | proteolysis | | | | |
| L1A4 | H108DE | liab: DS | H-CDR3 | low | 0.0 | yes |
| | | isomerization | | | | |
| L1A4 | H108DS | liab: DS | H-CDR3 | low | +1.0 | no |
| | | isomerization | | | | |
| L1A4 | H108DA | liab: DS | H-CDR3 | low | +1.0 | no |
| | | isomerization | | | | |
| L1A4 | H112WY | liab: Trp | H-CDR3 | low | 0.0 | no |
| | | oxidation | | | | |
| L1A4 | H112WF | liab: Trp | H-CDR3 | low | 0.0 | no |
| | | oxidation | | | | |
| L1A4 | H122WL | germlining | H-FW4 | medium | 0.0 | no |
| L1A4 | H123IV | germlining | H-FW4 | medium | 0.0 | yes |
| L1A4 | H124RT | germlining | H-FW4 | medium | −1.0 | yes |
| L1A4 | H126AS | germlining | H-FW4 | medium | 0.0 | no |
| L1A4 | H127PS | germlining | H-FW4 | medium | 0.0 | yes |
| L1A4 | L2CS | liab: cys & | L-FW1 | high | 0.0 | yes |
| | | germlining | | | | |
| L1A4 | L2CA | liab: cys | L-FW1 | high | 0.0 | no |
| L1A4 | L3VA | germlining | L-FW1 | medium | 0.0 | yes |
| L1A4 | L32CY | liab: cys & | L-FW2 | high | 0.0 | no |
| | | germlining | | | | |
| L1A4 | L32CA | liab: cys | L-FW2 | high | 0.0 | no |
| L1A4 | L32CV | liab: cys | L-FW2 | high | 0.0 | no |
| L1A4 | L32CL | liab: cys | L-FW2 | high | 0.0 | no |
| L1A4 | L32CI | liab: cys | L-FW2 | high | 0.0 | no |
| L1A4 | L34HQ | germlining | L-FW2 | medium | −0.5 | yes |
| L1A4 | L38NK | germlining | L-FW2 | medium | +1.0 | no |
| L1A4 | L43LM | germlining | L-FW2 | medium | 0.0 | no |
| L1A4 | L44LI | germlining | L-FW2 | medium | 0.0 | yes |
| L1A4 | L49KQ | liab: | L-CDR2 | medium | −1.0 | no |
| | | proteolysis | | | | |
| L1A4 | L49KS | liab: | L-CDR2 | medium | −1.0 | no |
| | | proteolysis | | | | |
| L1A4 | L49KA | liab: | L-CDR2 | medium | −1.0 | no |
| | | proteolysis | | | | |
| L1A4 | L50RQ | liab: | L-CDR2 | medium | −1.0 | no |
| | | proteolysis | | | | |
| L1A4 | L50RS | liab: | L-CDR2 | medium | −1.0 | no |
| | | proteolysis | | | | |
| L1A4 | L50RA | liab: | L-CDR2 | medium | −1.0 | no |
| | | proteolysis | | | | |
| L1A4 | L54IV | germlining | L-FW3 | medium | 0.0 | yes |
| L1A4 | L62RK | germlining | L-FW3 | medium | 0.0 | no |
| L1A4 | L76PA | germlining | L-FW3 | medium | 0.0 | yes |
| L1A4 | L83FY | germlining | L-FW3 | medium | 0.0 | no |
| L1A4 | L85WY | liab: Trp | L-CDR3 | low | 0.0 | no |
| | | oxidation | | | | |
| L1A4 | L85WF | liab: Trp | L-CDR3 | low | 0.0 | no |
| | | oxidation | | | | |
| L1A4 | L96VL | germlining | L-FW4 | medium | 0.0 | no |
| L1A4 | L97LT | germlining | L-FW4 | medium | 0.0 | yes |

TABLE 8
L2A1 variable positions

| | | | | Risk of | Change |
|---|---|---|---|---|---|
| | | | | leaving | in |
| Parent | Mutation | Purpose(s) | Location | as native | charge |
| L2A1 | HOHQ | germlining | H-FW1 | medium | −0.5 |
| L2A1 | H8GA | germlining | H-FW1 | medium | 0.0 |
| L2A1 | H9GE | germlining | H-FW1 | medium | −1.0 |
| L2A1 | H13IP | germlining | H-FW1 | medium | 0.0 |
| L2A1 | H16AS | germlining | H-FW1 | medium | 0.0 |
| L2A1 | H18RK | germlining | H-FW1 | medium | 0.0 |
| L2A1 | H19IV | germlining | H-FW1 | medium | 0.0 |
| L2A1 | H22EK | germlining | H-FW1 | medium | +2.0 |
| L2A1 | H23VA | germlining | H-FW1 | medium | 0.0 |
| L2A1 | H24TS | germlining | H-FW1 | medium | 0.0 |
| L2A1 | H27KN | liab: Lys | H-CDR1 | low | −1.0 |
| | | glycation | | | |
| L2A1 | H27KR | liab: Lys | H-CDR1 | low | 0.0 |
| | | glycation | | | |
| L2A1 | H27KQ | liab: Lys | H-CDR1 | low | −1.0 |
| | | glycation | | | |
| L2A1 | H27KS | liab: Lys | H-CDR1 | low | −1.0 |
| | | glycation | | | |
| L2A1 | H27KA | liab: Lys | H-CDR1 | low | −1.0 |
| | | glycation | | | |
| L2A1 | H29MQ | liab: Met | H-CDR1 | low | 0.0 |
| | | oxidation | | | |
| L2A1 | H29ML | liab: Met | H-CDR1 | low | 0.0 |
| | | oxidation | | | |
| L2A1 | H29MS | liab: Met | H-CDR1 | low | 0.0 |
| | | oxidation | | | |
| L2A1 | H29MA | liab: Met | H-CDR1 | low | 0.0 |
| | | oxidation | | | |
| L2A1 | H49WY | liab: Trp | H-CDR2 | low | 0.0 |
| | | oxidation | | | |
| L2A1 | H49WF | liab: Trp | H-CDR2 | low | 0.0 |
| | | oxidation | | | |
| L2A1 | H50MI | liab: Met | H-CDR2 | low | 0.0 |
| | | oxidation | | | |
| L2A1 | H50MQ | liab: Met | H-CDR2 | low | 0.0 |
| | | oxidation | | | |
| L2A1 | H50ML | liab: Met | H-CDR2 | low | 0.0 |
| | | oxidation | | | |
| L2A1 | H50MS | liab: Met | H-CDR2 | low | 0.0 |
| | | oxidation | | | |
| L2A1 | H50MA | liab: Met | H-CDR2 | low | 0.0 |
| | | oxidation | | | |
| L2A1 | H58NQ | liab: Nglyco | H-CDR2 | high | 0.0 |
| L2A1 | H58ND | liab: Nglyco | H-CDR2 | high | −1.0 |
| L2A1 | H58NS | liab: Nglyco | H-CDR2 | high | 0.0 |
| L2A1 | H58NA | liab: Nglyco | H-CDR2 | high | 0.0 |
| L2A1 | H60SA | liab: Nglyco | H-CDR2 | high | 0.0 |
| L2A1 | H60SN | liab: Nglyco | H-CDR2 | high | 0.0 |
| L2A1 | H61WQ | liab: Trp | H-CDR2 | low | 0.0 |
| | | oxidation | | | |
| L2A1 | H61WY | liab: Trp | H-CDR2 | low | 0.0 |
| | | oxidation | | | |
| L2A1 | H61WF | liab: Trp | H-CDR2 | low | 0.0 |
| | | oxidation | | | |
| L2A1 | H73MT | germlining | H-FW3 | medium | 0.0 |
| L2A1 | H74DS | germlining | H-FW3 | medium | +1.0 |
| L2A1 | H75TI | germlining | H-FW3 | medium | 0.0 |
| L2A1 | H76ES | germlining | H-FW3 | medium | +1.0 |
| L2A1 | H79FY | germlining | H-FW3 | medium | 0.0 |
| L2A1 | H83RS | germlining | H-FW3 | medium | −1.0 |
| L2A1 | H84GR | germlining | H-FW3 | medium | +1.0 |
| L2A1 | H87VS | germlining | H-FW3 | medium | 0.0 |
| L2A1 | H121VT | germlining | H-FW4 | medium | 0.0 |
| L2A1 | H122RL | germlining | H-FW4 | medium | −1.0 |
| L2A1 | H124VT | germlining | H-FW4 | medium | 0.0 |
| L2A1 | L1AQ | germlining | L-FW1 | medium | 0.0 |
| L2A1 | L10MV | germlining | L-FW1 | medium | 0.0 |
| L2A1 | L12AG | germlining | L-FW1 | medium | 0.0 |
| L2A1 | L32FY | germlining | L-FW2 | medium | 0.0 |
| L2A1 | L35YH | germlining | L-FW2 | medium | +0.5 |
| L2A1 | L39PA | germlining | L-FW2 | medium | 0.0 |
| L2A1 | L43IM | germlining | L-FW2 | medium | 0.0 |
| L2A1 | L45FY | germlining | L-FW2 | medium | 0.0 |
| L2A1 | L46DE | liab: DD | L-CDR2 | low | 0.0 |
| | | isomerization | | | |
| L2A1 | L46DS | liab: DD | L-CDR2 | low | +1.0 |
| | | isomerization | | | |
| L2A1 | L46DA | liab: DD | L-CDR2 | low | +1.0 |
| | | isomerization | | | |
| L2A1 | L47DE | liab: DD | L-CDR2 | low | 0.0 |
| | | isomerization | | | |
| L2A1 | L47DS | liab: DD | L-CDR2 | low | +1.0 |
| | | isomerization | | | |
| L2A1 | L47DA | liab: DD | L-CDR2 | low | +1.0 |
| | | isomerization | | | |
| L2A1 | L48DE | liab: DD | L-CDR2 | low | 0.0 |
| | | isomerization | | | |
| L2A1 | L48DS | liab: DD | L-CDR2 | low | +1.0 |
| | | isomerization | | | |
| L2A1 | L48DA | liab: DD | L-CDR2 | low | +1.0 |
| | | isomerization | | | |
| L2A1 | L49KQ | liab: | L-CDR2 | medium | −1.0 |
| | | proteolysis | | | |
| L2A1 | L49KS | liab: | L-CDR2 | medium | −1.0 |
| | | proteolysis | | | |
| L2A1 | L49KA | liab: | L-CDR2 | medium | −1.0 |
| | | proteolysis | | | |
| L2A1 | L50RQ | liab: | L-CDR2 | medium | −1.0 |
| | | proteolysis | | | |
| L2A1 | L50RS | liab: | L-CDR2 | medium | −1.0 |
| | | proteolysis | | | |
| L2A1 | L50RA | liab: | L-CDR2 | medium | −1.0 |
| | | proteolysis | | | |
| L2A1 | L56SD | germlining | L-FW3 | medium | −1.0 |
| L2A1 | L60AG | germlining | L-FW3 | medium | 0.0 |
| L2A1 | L62RK | germlining | L-FW3 | medium | 0.0 |
| L2A1 | L63PS | germlining | L-FW3 | medium | 0.0 |
| L2A1 | L65DN | germlining | L-FW3 | medium | +1.0 |
| L2A1 | L73NG | germlining | L-FW3 | medium | 0.0 |
| L2A1 | L74VL | germlining | L-FW3 | medium | 0.0 |
| L2A1 | L76PA | germlining | L-FW3 | medium | 0.0 |
| L2A1 | L81TD | germlining | L-FW3 | medium | −1.0 |
| L2A1 | L83IY | germlining | L-FW3 | medium | 0.0 |
| L2A1 | L85NQ | liab: NT | L-CDR3 | low | 0.0 |
| | | deamidation | | | |
| L2A1 | L85NS | liab: NT | L-CDR3 | low | 0.0 |
| | | deamidation | | | |
| L2A1 | L85NA | liab: NT | L-CDR3 | low | 0.0 |
| | | deamidation | | | |
| L2A1 | L85NW | liab: NT | L-CDR3 | low | 0.0 |
| | | deamidation | | | |
| L2A1 | L95RK | germlining | L-FW4 | medium | 0.0 |

### Example 2. Neutralization Analysis of L2A1 Variant Antibodies Evaluated Against a Panel of HIV-1 Viruses

Variant L2A1 antibodies (Table 9) were evaluated for neutralization activity.

TABLE 9
Complete variable heavy and light chain
sequences for L2A1 variants

| Designation | VH amino acid sequence | VL amino acid sequence |
|---|---|---|
| L2A1_H58NQ | HVQLVQSGGGVKKIGAAVRIS | ASALTQPASMSASPGQ |
| | CEVTGYKFMDQLINWVRQAP | SVTISCSGTRHIISAWFQ |
| | GQGLEWMGWMNPTYGQVQ | QYPGKPPKLIIFDDDKR |
| | YSWRFEGRVTMTRDMDTETA | PSGVPSRFSASRPGDTA |
| | FMELRGLRVDDTAVYYCARG | SLTISNVQPEDEATYIC |
| | PSGENYPFHYWGQGVRVVVSS | NTYEFFGGGTRLTVL |
| | (SEQ ID NO: 7) | (SEQ ID NO: 6) |
| L2A1_H58ND | HVQLVQSGGGVKKIGAAVRIS | ASALTQPASMSASPGQ |
| | CEVTGYKFMDQLINWVRQAP | SVTISCSGTRHIISAWFQ |
| | GQGLEWMGWMNPTYGQVD | QYPGKPPKLIIFDDDKR |
| | YSWRFEGRVTMTRDMDTETA | PSGVPSRFSASRPGDTA |
| | FMELRGLRVDDTAVYYCARG | SLTISNVQPEDEATYIC |
| | PSGENYPFHYWGQGVRVVVSS | NTYEFFGGGTRLTVL |
| | (SEQ ID NO: 8) | (SEQ ID NO: 6) |
| L2A1_H58NS | HVQLVQSGGGVKKIGAAVRIS | ASALTQPASMSASPGQ |
| | CEVTGYKFMDQLINWVRQAP | SVTISCSGTRHIISAWFQ |
| | GQGLEWMGWMNPTYGQVSY | QYPGKPPKLIIFDDDKR |
| | SWRFEGRVTMTRDMDTETAF | PSGVPSRFSASRPGDTA |
| | MELRGLRVDDTAVYYCARGP | SLTISNVQPEDEATYIC |
| | SGENYPFHYWGQGVRVVVSS | NTYEFFGGGTRLTVL |
| | (SEQ ID NO: 9) | (SEQ ID NO: 6) |
| L2A1_H58NA | HVQLVQSGGGVKKIGAAVRIS | ASALTQPASMSASPGQ |
| | CEVTGYKFMDQLINWVRQAP | SVTISCSGTRHIISAWFQ |
| | GQGLEWMGWMNPTYGQVA | QYPGKPPKLIIFDDDKR |
| | YSWRFEGRVTMTRDMDTETA | PSGVPSRFSASRPGDTA |
| | FMELRGLRVDDTAVYYCARG | SLTISNVQPEDEATYIC |
| | PSGENYPFHYWGQGVRVVVSS | NTYEFFGGGTRLTVL |
| | (SEQ ID NO: 10) | (SEQ ID NO: 6) |
| L2A1_H60SA | HVQLVQSGGGVKKIGAAVRIS | ASALTQPASMSASPGQ |
| | CEVTGYKFMDQLINWVRQAP | SVTISCSGTRHIISAWFQ |
| | GQGLEWMGWMNPTYGQVN | QYPGKPPKLIIFDDDKR |
| | YAWRFEGRVTMTRDMDTET | PSGVPSRFSASRPGDTA |
| | AFMELRGLRVDDTAVYYCAR | SLTISNVQPEDEATYIC |
| | GPSGENYPFHYWGQGVRVVVSS | NTYEFFGGGTRLTVL |
| | (SEQ ID NO: 11) | (SEQ ID NO: 6) |
| L2A1_H60SN | HVQLVQSGGGVKKIGAAVRIS | ASALTQPASMSASPGQ |
| | CEVTGYKFMDQLINWVRQAP | SVTISCSGTRHIISAWFQ |
| | GQGLEWMGWMNPTYGQVN | QYPGKPPKLIIFDDDKR |
| | YNWRFEGRVTMTRDMDTET | PSGVPSRFSASRPGDTA |
| | AFMELRGLRVDDTAVYYCAR | SLTISNVQPEDEATYIC |
| | GPSGENYPFHYWGQGVRVVVSS | NTYEFFGGGTRLTVL |
| | (SEQ ID NO: 12) | (SEQ ID NO: 6) |

Neutralization activity was assessed againt a panel of 6 HIV-1 viruses (DU172.17, CAP45.2.00.G3, CNE20, REJ04541.67, AC10.0.29, and WIT04160.33, Table 10). Envelope (env) sequences were cloned into replication-competent infectious molecular clones (IMCs) carrying a Tat-regulated Renilla Luc reporter gene (Env.IMC.LucR). Antibodies were diluted to 50 μg/mL and then serially diluted 4-fold across 8 dilutions before being mixed with virus. The antibody-virus mixture was subsequently used to infected TZM-BL reporter cells. Reduction in luciferase expression was used to assess antibody-mediated viral neutralization. For each antibody dilution, neutralization was reported as the percent reduction in luciferase as compared to virus-only negative control. Antibody neutralization titers were calculated using a sigmoidal dose response curve in Graphpad Prism and reported as the antibody concentration required to inhibit 50% of the viral infection (IC₅₀). Antibody IC₅₀ values are derived from a single assay wherein each antibody was run in duplicate.

TABLE 10
Panel of HIV-1 viruses

| Virus | HIV-1 Clade |
|---|---|
| AC10.0.29 | B |
| REJO4541.67 | B |
| WITO4160.33 | B |
| DU172.17 | C |
| CAP45.2.00.G3 | C |
| CNE20 | CRF07 |

To assess whether modifications to L2A1 affected neutralization activity, we measured IC₅₀ values for the 6 variants against a panel of six HIV-1 IMCs with a range of previously reported sensitivity to the parent (L2A1) and control (3BNC117) antibodies. Both the parent and antibody variants were expressed from HEK-293 cells using transient transfection and purified with protein A affinity chromatography. In addition to these 7 antibodies, we also tested 3BNC117, a well-characterized CD4-binding site antibody as a positive control and a non-HIV specific human IgG1 antibody as a negative control.

The modifications evaluated in this dataset were designed to disrupt the consensus N-linked glycosylation sequence motif in H-CDR2 (H58N-H59Y-H60S). Six different variants distributed across two amino acid positions were tested. The first 4 variants were designed to eliminate the N-linked glycosylation site by modifying the asparagine at position H58 to various different residues, including a glutamine, “L2A1_H58NQ”, an aspartic acid “L2A1_H58ND”, a serine “L2A1-H58NS” and an alanine “L2A1_H58NA”. The 5th and 6th variants eliminated the N-linked glycosylation site by modifying the serine at position 60 to either an alanine “L2A1_H60SA” or an asparagine, “L2A1_H60SN”. The initial set of 4 antibodies containing a mutation to the asparagine at position 58 all demonstrated reduced breadth (60%-83%) as compared to parent antibody's (L2A1) breadth (100%, Table 11). None of the four variants containing a mutation at position H58N demonstrated neutralizing activity against HIv-1 virus CNE20, and two of the variants, “L2A1_H58NQ” and “L2A1_H58ND” also lost neutralizing activity against a second virus, AC10.0.29 (Table 11). In contrast, the third and fourth antibodies containing less bulky mutations to a serine (H58NS) and an alanine (H58NA), respectively, demonstrated similar neutralizing activity against the AC10.0.29 virus as the parent mAb L2A1. The 5^{th} and 6^{th} antibodies, which contained mutations to the serine at position 60, “L2A1_H60SA” and “L2A1_H60SN” neutralized 100% of viruses tested with a similar geometric mean IC₅₀ as parent mAb L2A1 (0.086 μg/mL and 0.100 μg/mL, respectively, vs 0.093 μg/mL (L2A1), Table 11). In summary, two sets of modifications were proposed to eliminate the N-linked glycosylation site in H-CDR2 of L2A1. Mutations modifying the serine at position 60 had no measurable impact on parent antibody breadth or potency, while mutations to the asparagine at position 58 abrogated neutralizing activity against 1-2 HIV-1 viruses.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference for the purposes in the context of which they are cited.

TABLE 11
(IC₅₀ μg/mL)

| | | | | | | | Geometric mean | Breadth |
|---|---|---|---|---|---|---|---|---|
| Designation | DU172.17 | CAP45.2.00.G3 | CNE20 | REJO4541.67 | WITO4160.33 | AC10.0.29 | (IC₅₀ μg/mL)* | (%) [symbol] |
| L2A1 | 0.128 | 0.069 | 0.050 | 0.022 | 0.213 | 0.306 | 0.093 | 100% |
| L2A1_H58NQ | 2.271 | IND | >50 | 0.025 | 0.222 | >50 | 0.231 | 60% |
| L2A1_H58ND | 20.938 | IND | >50 | 0.212 | 0.650 | >50 | 1.424 | 60% |
| L2A1_H58NS | 0.102 | 0.040 | >50 | 0.206 | 0.182 | 0.091 | 0.107 | 83% |
| L2A1_H58NA | 0.218 | 0.050 | >50 | 0.104 | 0.259 | 0.098 | 0.124 | 83% |
| L2A1_H60SA | 0.056 | 0.039 | 0.170 | 0.105 | 0.148 | 0.072 | 0.086 | 100% |
| L2A1_H60SN | 0.044 | 0.032 | 0.317 | 0.121 | 0.212 | 0.087 | 0.100 | 100% |
| 3BNC117 | 1.255 | 2.704 | >50 | 0.070 | 0.053 | 11.002 | 1.275 | 83% |

*Geometric mean IC50 is calculated using only the values from the neutralized viruses (IC₅₀ ≤ 50 μg/mL). [symbol]
Breadth calculated as the fraction of viruses which are neutralized at IC₅₀ values ≤ 50 μg/mL.
IND = indeterminate

What is claimed is:

1. An anti-HIV antibody comprising a heavy chain variable (V_{H}) region and a light chain variable (V_{L}) region, wherein:
(a) the V_{H} region comprises a CDR1 sequence, a CDR2, sequence, and a CDR3 sequence as set forth in Table 2 for the antibody designated L1A1 in which at least one of CDR2 or CDR3 comprises at least one substitution, wherein the substitution is selected from the group consisting of Y or F at position 49; I, Q, L, S, or A at position 50; D, S, A, or Q at position 101, W A, or N at position 103; Q, S, or A at position 105; Q, S or A at position 106; Q, S, or A at position 107; and Y or F at position 113; said positions determined with reference to SEQ ID NO:1; and
(b) the V_{L} region comprises: a CDR1 sequence, a CDR2, sequence, and a CDR3 sequence as set forth in Table 2 for the antibody designated L1A1; or at least one substitution in the CDR2 sequence, or CDR3 sequence, wherein the at least one substitution is selected from the group consisting of Q, S, or A, at position 49, Q, S, or A at position 50; and Y or F at position 85, said positions determined with reference to SEQ ID NO:2.

2. An anti-HIV antibody comprising a heavy chain variable (V_{H}) region and a light chain variable (V_{L}) region, wherein:
a) the V_{H} region comprises a CDR1 sequence, a CDR2, sequence, and a CDR3 sequence as set forth in Table 2 for the antibody designated L1A1; or comprises at least one substitution in the CDR2 or CDR3, wherein the substitution is selected from the group consisting of Y or F at position 49; I, Q, L, S, or A at position 50; D, S, A, or Q at position 101, W A, or N at position 103; Q, S, or A at position 105; Q, S or A at position 106; Q, S, or A at position 107; and Y or F at position 113; said positions determined with reference to SEQ ID NO:1; and
(b) the V_{L} region comprises a CDR1 sequence, a CDR2, sequence, and a CDR3 sequence as set forth in Table 2 for the antibody designated L1A1 in which the CDR2 or CDR3 comprises at least one substitution, wherein the at least one substitution is selected from the group consisting of Q, S, or A, at position 49, Q, S, or A at position 50; and Y or F at position 85, said positions determined with reference to SEQ ID NO:2.

3. The anti-HIV antibody of claim 1, wherein the V_{H} region comprises at least one of the following, as numbered with reference to SEQ ID NO:1: V at position 1, Q at position 2, V at position 4, E at position 9, V at position 10, K at position 12, P at position 13, K at position 18, K at position 22, S at position 24, V at position 36, A at position 39, P at position 40, Q a position 42, M at position 47; R a position 66, I at position 75; S at position 76, M at position 80; E at position 81, L at position 82; S at position 83; R at position 84; R at position 86; S at position 87; L at position 123; V at position 124; or S at position 127; and/or
the V_{L} region comprises at least one of the following, as numbered with reference to SEQ ID NO:2: G at position 12; Y, A, V, L, or I at position 32; H at position 35; K at position 38; M at position 43, K at position 62, E at position 77; A at position 80; D at position 81, Y at position 83, or F at position 90.

4. The anti-HIV antibody of claim 1, wherein the V_{H} region has at least 70% identity to SEQ ID NO:1; and/or the V_{L} region has at least 70% identity to SEQ ID NO:2.

5. An anti-HIV antibody comprising a heavy chain variable (V_{H}) region and a light chain variable (V_{L}) region, wherein:
(a) the V_{H} region comprises a CDR1 sequence, a CDR2, sequence, and a CDR3 sequence as set forth in Table 2 for the antibody designated L1A4 in which at least one of CDR2 or CDR3 comprises at least one substitution, wherein the substitution is selected from the group consisting of Y or F at position 49; I, Q, L, S, or A at position 50; E, S, or A at position 98; R or K at position 99; E, S, or A at position 101, A or S at position 102; Q, S, A, or G at position 104; Q, S, or A at position 105; E, S, or A at position 108; and Y or F at position 112; said positions determined with reference to SEQ ID NO:3; and
(b) the V_{L} region comprises: a CDR1 sequence, a CDR2, sequence, and a CDR3 sequence as set forth in Table 2 for the antibody designated L1A4; or at least one substitution in the CDR2 sequence, or CDR3 sequence, wherein the at least one substitution is selected from the group consisting of Q, S, or A, at position 49; Q, S, or A at position 50; and Y or F at position 85, said positions determined with reference to SEQ ID NO:4.

6. An anti-HIV comprising a heavy chain variable (V_{H}) region and a light chain variable (V_{L}) region, wherein:
a) the V_{H} region comprises a CDR1 sequence, a CDR2, sequence, and a CDR3 sequence as set forth in Table 2 for the antibody designated L1A4; or comprises at least one substitution in the CDR2 or CDR3, wherein the substitution is selected from the group consisting of Y or F at position 49; I, Q, L, S, or A at position 50; E, S, or A at position 98; R or K at position 99; E, S, or A at position 101, A or S at position 102; Q, S, A, or G at position 104; Q, S, or A at position 105; E, S, or A at position 108; and Y or F at position 112; said positions determined with reference to SEQ ID NO:3; and
(b) the V_{L} region comprises: a CDR1 sequence, a CDR2, sequence, and a CDR3 sequence as set forth in Table 2 for the antibody designated L1A4 comprising at least one substitution in the CDR2 sequence, or CDR3 sequence, wherein the at least one substitution is selected from the group consisting of Q, S, or A, at position 49; Q, S, or A at position 50; and Y or F at position 85, said positions determined with reference to SEQ ID NO:4.

7. The anti-HIV antibody of claim 5, wherein the V_{H} comprises at least one of the following substitutions as numbered with reference to SEQ ID NO:3: V at position 1, Q at position 2, E at position 9, A at position 15, K position 22, S at position 24, V at position 36, T at position 68, T at position 73, S at position 74, I at position 75, S at position 76, Y at position 79, M at position 80, L at position 82, S at position 83, R at position 84, R at position 86, S at position 87, A at position 91, V at position 92, L at position 122, V at position 123, T at position 124, S at position 126, or S at position 127; and/or
the V_{L} comprises at least one of the follow substitutions as numbered with reference to SEQ ID NO:4: S or A at position 2; A at position 3; Y, A, V, L, or I at position 32; Q at position 34, K at position 38, M at position 43, I at position 44, V at position 54, K at position 62, A at position 76, Y at position 83, L at position 96, or T at position 97.

8. The anti-HIV antibody of claim 5, wherein the V_{H} comprises an amino acid sequence having at least 80% identity to SEQ ID NO:3; and/or the V_{L} region comprises an amino acid sequence having at least 80% identity to SEQ ID NO:4.

9. An anti-HIV antibody comprising a heavy chain variable (V_{H}) region and a light chain variable (V_{L}) region, wherein:
(a) the V_{H} region comprises a CDR1 sequence, a CDR2, sequence, and a CDR3 sequence as set forth in Table 2 for the antibody designated L2A1 in which at least one of CDR1 or CDR2 comprises at least one substitution, wherein the substitution is selected from the group consisting of N, R, Q, S, or A at position 27; Q, L, S or A at position 29; Y or F at position 49; I, Q, L, S, or A at position 50; Q, D, S or A at position 58; A or N at position 60; and Q, Y, or F at position 61; said positions determined with reference to SEQ ID NO:5; and
(b) the V_{L} region comprises: a CDR1 sequence, a CDR2, sequence, and a CDR3 sequence as set forth in Table 2 for the antibody designated L2A1; or at least one substitution in the CDR2 sequence, or CDR3 sequence, wherein the at least one substitution is selected from the group consisting of E, S, or A at position 46; E, S, or A at position 47; E, S, or A at position 48; Q, S, or A at position 49; Q, S, or A at position 50; and Q, S, A or W at position 85, said positions determined with reference to SEQ ID NO:6.

10. An anti-HIV antibody comprising a heavy chain variable (V_{H}) region and a light chain variable (V_{L}) region, wherein:
a) the V_{H} region comprises a CDR1 sequence, a CDR2, sequence, and a CDR3 sequence as set forth in Table 2 for the antibody designated L2A1; or comprises at least one substitution in the CDR1 or CDR2, wherein the substitution is selected from the group consisting of N, R, Q, S, or A at position 27; Q, L, S or A at position 29; Y or F at position 49; I, Q, L, S, or A at position 50; Q, D, S or A at position 58; A or N at position 60; and Q, Y, or F at position 61; said positions determined with reference to SEQ ID NO:5; and
(b) the V_{L} region comprises: a CDR1 sequence, a CDR2, sequence, and a CDR3 sequence as set forth in Table 2 for the antibody designated L2A1 in which the CDR2 sequence, or CDR3 sequence comprises at least one substitution, wherein the at least one substitution is selected from the group consisting of E, S, or A at position 46; E, S, or A at position 47; E, S, or A at position 48; Q, S, or A at position 49; Q, S, or A at position 50; and Q, S, A or W at position 85, said positions determined with reference to SEQ ID NO:6.

11. The anti-HIV antibody of claim 9, wherein the V_{H} comprises at least one of the following substitutions as numbered with reference to SEQ ID NO:5: A at position 8, E at position 9, P at position 13, S at position 16, K at position 18; V at position 19, K at position 22, A at position 23, S at position 24, T position 73, S at position 74, I at position 75, S at position 76, Y at position 79, S at position 83, R at position 84, S at position 87, T at position 121, L at position 122, or T at position 124; and/or
the V_{L} comprises at least one of the follow substitutions as numbered with reference to SEQ ID NO:6: Q at position 1, V at position 10, G at position 12, Y at position 32, H at position 35, A at position 39, M at position 43, Y at position 45, D at position 56, G at position 60, K at position 62, S at position 63, N at position 65, G at position 73, L at position 74, A at position 76, D at position 81, Y at position 83, or K at position 95.

12. The anti-HIV antibody of claim 9, wherein the V_{H} comprises the CDR1, CDR2, and CDR3 of SEQ ID NO:11 or SEQ ID NO:12.

13. The anti-HIV antibody of claim 9, wherein the V_{H} comprises an amino acid sequence having at least 80% identity to SEQ ID NO:5; and/or the V_{L} region comprises an amino acid sequence having at least 80% identity to SEQ ID NO:6.

14. An expression vector comprising a polynucleotide encoding the V_{H} region and/or the V_{L} region of the anti-HIV antibody of claim 1.

15. A host cell that comprises an expression vector of claim 14.

16. A host cell comprising a polynucleotide that encodes the V_{H} region and/or the V_{L} region of the anti-HIV antibody of claim 1.

17. A method of treating or preventing an HIV-1 infection, the method comprising administering the anti-HIV antibody of claim 1 to a patient that is infected with an HIV-1 virus, or is at risk of infection of with an HIV-1 virus.

18. The method of claim 17, further comprising administering a latency reversing agent to the patient.
